SEQUENCE LISTING (1 sequence)

SEQ ID NO: 1 - DNA, 34 bp, synthetic construct
  source 1..34: mol_type = other DNA
gcctaggcagctatgaccatgattacgccaagcg

(19) United States
(12) Patent Application Publication
(10) Pub. No.: US 2026/0234576 A1
(43) Pub. Date: Aug. 13, 2026

(54) PHOSPHORYLATION-PROTEIN COMPLEMENTATION ASSAY AND RELATED SYSTEMS AND METHODS

(71) Applicant: California Institute of Technology, Pasadena, CA (US)

(72) Inventors: David A. Van Valen, Pasadena, CA (US); Changhua Yu, Pasadena, CA (US); Edward Pao, Pasadena, CA (US)

(21) Appl. No.: 19/363,588

(22) Filed: Oct. 20, 2025

Related U.S. Application Data
(60) Provisional application No. 63/709,603, filed on Oct. 21, 2024.

(51) Int. Cl. C12N 9/12; C12N 15/10

(52) U.S. Cl. CPC C12N 9/12; C12N 15/1065; C12N 15/1086; C12Y 207/10002

(57) ABSTRACT

Provided herein include a phosphorylation-protein complementation reporter system, an abundance-protein complementation reporter systems, and assays and methods of using the same to detect phosphorylation and for high-throughput kinase variant screening. In some embodiments, the phosphorylation-protein complementation reporter system comprises a first fusion protein comprising a kinase substrate linked to (i) a kinase domain and (ii) a first segment of a reporter molecule; and a second fusion protein comprising a phosphor-amino-acid binding domain (PAABD) linked to a second segment of the reporter molecule, wherein the first segment and the second segment of the reporter molecule are capable of complementing one another to form an active reporter molecule via the interaction between the PAABD and the kinase substrate upon phosphorylation by the kinase domain.

### RELATED APPLICATIONS

This application claims the benefit under 35 U.S.C. § 119(e) of U.S. Provisional Patent Application Ser. No. 63/709,603, filed on Oct. 21, 2024, the content of this related application is incorporated herein by reference in its entirety for all purposes.

### STATEMENT REGARDING FEDERALLY SPONSORED R&D

This invention was made with government support under Grant No. DP2-GM149556 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810016-US_SequenceListing, created Oct. 20, 2025, which is 1,798 bytes in size. The information in the electronic format of the Sequence Listing is incorporated herein by reference in its entirety.

### BACKGROUND

### Field

The present disclosure relates generally to the field of molecular biology.

### Description of the Related Art

Kinases are proteins that regulate enzymes and biological signaling by phosphorylating other proteins. Disruption in kinase activity often results in disease states and as such the study of kinase regulation is key to finding potential treatments for these diseases. One key aspect of this regulation is the allosteric control of activity, where regions outside of a kinases' active site can control the stability, specificity of target, and activity of the kinase through small molecule binding or mutations. However, these allosteric sites are not well defined and are relatively difficult to discover. Current methodologies to find these allosteric sites are time and labor intensive, usually involving computational modeling, NMR, or x-ray crystallography and experimental validation.

An alternative method of allosteric site discovery is deep mutational scanning where every amino acid in regions distal to the kinase active site are systematically mutated to the other 19 amino acids. The output of this scan results in several variants of the same kinase with different activity levels. However, there still remains a need for methods and systems that can simultaneously measure the activity of the resulting variants in an efficient manner.

### SUMMARY

Provided herein includes a phosphorylation-protein complementation reporter system. The phosphorylation-protein complementation reporter system can, for example, comprise: a first fusion protein comprising a kinase substrate linked to (i) a kinase domain and (ii) a first segment of a reporter molecule; and a second fusion protein comprising a phosphor-amino-acid binding domain (PAABD) linked to a second segment of the reporter molecule, wherein the first segment and the second segment of the reporter molecule are capable of complementing one another to form an active reporter molecule via the interaction between the PAABD and the kinase substrate upon phosphorylation by the kinase domain.

In some embodiments, the kinase domain is a tyrosine kinase or a portion thereof, or a serine-threonine kinase or a portion thereof. For example, the kinase domain can be a Src family kinase (SFK) or a portion thereof. The kinase domain can be, or comprise, Fyn, Lck, or c-Src. In some embodiments, the PAABD is a Src homology 2 domain, a PTB domain, a 14-3-3b protein domain, a WD40 domain, a WW domain, a forkhead-associated FHA domain, or a combination thereof. The substrate can comprise tyrosine, serine, threonine, or a combination thereof. In some embodiments, the reporter molecule is an inhibitor-resistance conferring protein. In some embodiments, the kinase substrate is linked to i) the kinase domain and (ii) the first segment of the reporter molecule via a linker. In some embodiments, the phosphor-amino-acid binding domain (PAABD) is linked to the second segment of the reporter molecule via a linker. The reporter molecule can be, for example, dihydrofolate reductase (DHFR). The DHFR can be, for example, a methotrexate-resistant DHFR.

Provided herein includes an expression vector. The expression vector can comprise, for example, a polynucleotide encoding one or more of the phosphorylation-protein complementation reporter systems disclosed herein. In some embodiments, the polynucleotides encoding the first fusion protein and the second fusion protein are on a single plasmid. In some embodiments, the kinase domain comprises a variation, optionally a substitution, a deletion, and/or an insertion, relative to its wild type sequence, and the polynucleotide further comprises a variant barcode associated with the variation. The variant barcode can be, for example, about 4-20 bp in length.

Provided herein includes an abundance protein complementation reporter system. The system can, for example, comprise: a fusion protein comprising a kinase substrate linked to (i) a kinase domain and (ii) a first segment of a reporter molecule; and a second segment of the reporter molecule, and wherein the first segment and the second segment of the reporter molecule are capable of complementing one another to form an active reporter molecule. In some embodiments, the second segment of the reporter molecule is expressed at a higher level than the fusion protein. In some embodiments, the kinase domain is a tyrosine kinase or a portion thereof or a serine-threonine kinase or a portion thereof, optionally the kinase domain is a Src family kinase (SFK) or a portion thereof. The kinase domain can be, or comprise, Fyn, Lck, or c-Src. The reporter molecule can be, or comprises, dihydrofolate reductase (DHFR). The DHFR can be, for example, a methotrexate-resistant DHFR.

Provided herein includes an expression vector. The expression vector can, in some embodiments, comprise a polynucleotide encoding one or more of the abundance protein complementation reporter systems disclosed herein. In some embodiments, the polynucleotides encoding the fusion protein and the second segment of the reporter molecule are on a single plasmid. In some embodiments, the kinase domain comprises a variation, optionally a substitution, a deletion, and/or an insertion, relative to its wild type sequence, and the polynucleotide further comprises a variant barcode associated with the variation. The variant barcode can be about 4-20 bp in length.

Also provided herein includes a method of detecting phosphorylation. The method can, for example, comprise: introducing into a cell an expression vector comprising a polynucleotide encoding one or more of the phosphorylation-protein complementation reporter systems disclosed herein; culturing the cell in a selective culture medium under a condition for a time period, wherein the selective culture medium contains an inhibitor that inhibits cell proliferation in the absence of the active reporter molecule; and detecting a cell proliferation signal, wherein the cell proliferation signal indicates phosphorylation of the kinase substrate by the kinase domain. The cell can be a yeast cell. In some embodiments, the reporter molecule is DHFR, and the inhibitor is methotrexate. In some embodiments, detecting the cell proliferation signal comprises measuring optical density of the selective culture medium. In some embodiments, the method further comprises generating a cell growth profile and/or a cell growth rate. In some embodiments, the method further comprises: introducing into a cell an expression vector encoding one or more of the abundance protein complementation reporter systems disclosed herein, culturing the cell in a selective culture medium under a condition for a time period, wherein the selective culture medium contains an inhibitor that inhibits cell proliferation in the absence of the active reporter molecule; and detecting an abundance cell signal. In some embodiments, the method comprises: applying a hierarchical Bayesian regression model to the cell proliferation signal and the abundance cell signal to decompose the cell proliferation signal into a sum of scores each representing kinase stability, kinase activity or substrate specificity.

In some embodiments, the cell proliferation signal is decomposed into a linear sum of (a) the abundance cell signal multiplied by an abundance effect slope parameter (captures how abundance affects phosphor-PCA scores for each specific kinase-substrate pair); (b) an intercept representing the baseline phosphorylation activity independent of abundance effects; (c) a score representing kinase activity effect; and (d) a score representing substrate specificity effect. In some embodiments, the method further comprises identifying one or more mutations, based on the decomposition of the cell proliferation signal, that (i) negatively impact kinase catalytic activity and negatively impact kinase stability; (ii) negatively impact kinase catalytic activity and have minimal or no effect on abundance; and/or (iii) positively impact kinase catalytic activity. In some embodiments, the method further comprises identifying, based on cell proliferation signal and the abundance cell signal, one or more potential allosteric sites coupled to substrate specificity and/or kinase activity.

Provided herein includes a method of screening kinase variants. The method, in some embodiments, comprises: generating a kinase variant library comprising a plurality of expression vectors each comprising a polynucleotide encoding a phosphorylation protein complementation reporter system of any one of claims 1-10, wherein the kinase domain comprises a variation and the polynucleotide comprises a variant barcode associated with the variation; introducing into a cell an expression vector of the plurality of expression vectors; culturing the cell in a selective culture medium under a condition for a time period, wherein the selective culture medium contains an inhibitor that inhibits cell proliferation in the absence of the active reporter molecule; extracting expression vectors from surviving cells; and determining the variation in the kinase domain by sequencing the extracted expression vectors.

In some embodiments, the method further comprises: generating a kinase variant library comprising a plurality of expression vectors each comprising a polynucleotide encoding an abundance protein complementation reporter system of any one of claims 15-20, wherein the kinase domain comprises a variation and the polynucleotide comprises a variant barcode associated with the variation; introducing into a cell an expression vector of the plurality of expression vectors; culturing the cell in a selective culture medium under a condition for a time period, wherein the selective culture medium contains an inhibitor that inhibits cell proliferation in the absence of the active reporter molecule; extracting expression vectors from surviving cells; and determining the variation in the kinase domain by sequencing the extracted expression vectors.

In some embodiments, the variation in the kinase domain is a substitution, insertion, and/or deletion relative to the wild type sequence of the kinase domain. In some embodiments, generating the kinase variant library is carried out using comprehensive saturation mutagenesis. In some embodiments, the kinase variant library comprises polynucleotides encoding kinase variants of a same wild type kinase. In some embodiments, the kinase variant library comprises polynucleotides encoding kinase variants of different wild type kinases. The polynucleotide can further comprise a kinase-specific barcode associated with each wild type kinase. In some embodiments, the kinase variant library comprises polynucleotides encoding different kinase substrates. In some embodiments, wherein determining the variation in the kinase domain comprises sequencing the variant barcode, optionally using short-read sequencing, and determining the variation in the kinase domain using a lookup table. In some embodiments, the method further comprises creating a lookup table containing correspondences between kinase domain variations and variant barcodes using long read sequencing. In some embodiments, the method further comprises determining a relative abundance for each variant barcode. The cell can be a yeast cell. The cells with a detected cell proliferation signal can be surviving cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B depict a diagram of kinase activity reporter system (FIG. 1A) and an exemplary experimental workflow.

FIG. 2 depicts exemplary workflows of a dual phosphorylation and abundance protein complementation assay design (panel A) and a barcoded phosphor-PCA assay design. The results demonstrate that phospho-PCA can enable deep mutational scanning of kinase domains to understand the sequence-to-function relationship for this enzyme category. Panel A: Phospho-PCA utilizes a growth-based biosensor to couple kinase-substrate interactions to cell growth. Kinase-substrate interactions reconstitute the DHFR enzyme, conferring resistance to methotrexate selection. Abundance-PCA can be performed with the same construct by removing the PAABD, providing a score of folding stability. Panel B: Deep mutational scanning of kinase domains with phospho-PCA is performed by cloning a kinase variant library into the biosensor construct and barcoding the variants with 16 bp-long barcodes. Long-read sequencing creates a lookup table linking kinase variants to barcodes; the wild-type amino acid, the position, and the mutated amino acid describe each variant. Once cloned, the library is transformed into budding yeast cells, and the activity of each variant for phosphor- and abundance-PCA is measured by quantifying cell growth in the presence of methotrexate with short-read sequencing.

FIG. 3 displays non-limiting exemplary embodiments showing phospho-PCA quantitatively couples kinase-substrate interactions to cell growth. Panel A: Biosensors containing interacting kinase-substrate pairs resist selection, while biosensors with non-interacting pairs display significantly reduced growth. Panels B and C: Quantitative measurement of Fyn, Lck, and c-Src substrate preferences for Fyn-sub, Lck-sub, and c-Src-sub with phospho-PCA. Growth rates were computationally extracted during the exponential phase of cell growth; scores among all substrates for each kinase were standardized by z-score normalization and then rescaled to range between 0 and 2. Kinase-substrate interaction strengths measured with phospho-PCA are consistent with prior measurements of substrate preferences. Panel D: Heatmaps for abundance- and phospho-PCA measurements for Fyn and the Fyn/Fyn-sub interaction, respectively.

FIG. 4 displays non-limiting exemplary data showing mutations in conserved regions impact abundance-PCA scores of SFKs. Panels A-B: Principal component analysis reveals three distinct responses of positions to mutations. Group 1 and group 3 positions prefer negatively or positively charged residues, respectively, while group 2 positions prefer hydrophobic residues. Panel C: Positional response categories overlaid on the kinase structure. Group 2 positions cluster at the hydrophobic core, while group 1 and group 3 positions are enriched at the kinase surface. Panel D: Functional annotations and mutational effects for the kinase domains of Fyn, Lck, and c-Src. The six rows display for each position the Dunbrack annotation, whether the amino acid property is conserved across the three SFKs, the overall mutational effect, the favored amino acid property, the wildtype amino acid and its category, and a functional annotation. Panel E: Gain-of-function and loss-of-function positions categorized by surface exposure, Dunbrack annotation, and functional annotation.

FIG. 5 displays non-limiting exemplary data from phosphor-PCA revealing activity-biasing mutations and the function of clinical variants. Panel A: Hierarchical Bayesian model schematic for decoupling phospho-PCA scores into abundance, kinase activity, and substrate specificity components. The model decomposes measured phospho-PCA signals (phospho-PCAi) into a linear combination of scaled abundance effects (αij×Abundance Scale×βij), kinase activity effects (Activityikl), and substrate specificity effects (Substrate specificityi j kl). Hyperpriors regulate baseline variability across model components, with indices representing kinase identity (i), substrate identity (j), position (k), and mutation (l). Panel B: Example decoupling results for the Fyn-Fyn substrate pair, showing separation of measured phospho-PCA scores into constituent abundance, kinase activity, and substrate specificity components. Heatmaps display normalized scores across all single amino acid mutations for each position along the kinase domain. The decoupling reveals mutations that specifically impact kinase activity versus substrate recognition. Gain-of-function and loss-of-function sites are largely conserved across the three SFKs. Panel C: Conserved gain-of-function mutation V195E reveals a well-tuned co-evolutionary network. Left: Fraction of conformations in BioEmu sampling showing interactions among co-evolving residues (Coulombic, salt bridge, and Van der Waals) for V195E and wildtype kinases. Right: Network diagram highlighting interactions among co-evolving residues, with V195E establishing new electrostatic interactions that stabilize active conformations. Panel D: ProteinMPNN-based conformational bias analysis identifies conserved mutations favoring different kinase conformations. The heatmap shows predicted conformational preferences for mutations across inactive DFG-out, inactive DFG-in, and active DFG-in states. Mutations that favor inactive conformations (cool colors) correlate with negative activity scores, while mutations favoring active conformations (warm colors) correspond to gain-of-function effects. Panel E: Clinical variant analysis using ClinVar classifications. The scatter plot shows abundance-PCA versus phospho-PCA scores colored by pathogenic effect. Benign variants cluster near the origin with near-zero scores, while pathogenic variants show significant negative effects in one or both assays. The corresponding bar chart displays mean feature values by pathogenic effect, demonstrating clear separation between benign and harmful variants. Panel F: Cross-analysis of CADD scores and experimental screening results reveals modes of likely pathogenic variants. Left: Scatter plot of gnomAD population variation with points colored by CADD score quantiles, showing that high CADD variants display reduced abundance and activity. Right: Mean feature values by CADDscore quantile demonstrate increasing negative effects with higher predicted pathogenicity scores, validating the correspondence between computational predictions and experimental measurements.

FIG. 6 (Panels A-D) displays non-limiting exemplary embodiments showing specificity determining variants and allosteric networks governing substrate specificity.

FIG. 7 depicts a table showing sequencing coverage for abundance- and phosphor-PCA libraries.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Protein kinases selectively transfer phosphate groups to their substrates, significantly affecting the behavior of the substrate protein by causing changes in enzymatic activity, cellular localization, and affinity to binding partners. Because protein kinase activity is regulated by upstream information, this enzyme category plays a key role in the transmission of information to various cellular processes. Given the central role that protein kinases play in the information processing capabilities of the cell, their ability to control their activity and choose a specific set of substrates is essential to their function.

Despite their importance to human biology, understanding of kinase domains is incomplete. One important knowledge gap is how genetic variants of protein kinases impact their function. Dysregulation of protein kinases, which can be caused by a single missense mutation, is an important driver of numerous diseases, including autoimmunity and cancer. How specific variants within kinase domains affect enzymatic activity and substrate recognition remains an open question. Recent advances in deep mutational scanning have begun to address this challenge by systematically profiling the functional consequences of thousands of mutations across kinase domains. However, most studies do not account for the impact of variants on kinase abundance; the impact of this confounding variable can be difficult to distinguish from enzymatic activity or substrate preference. Improved knowledge of how variants affect kinase function can reveal sites distal to the active site that govern enzymatic activity through allostery. Targeting divergent sites that allosterically communicate with the active site or substrate interface may be a path to generating potent and specific molecules that change kinase activity.

Another key gap is a mechanistic and quantitative understanding of how kinase domains contribute to substrate specificity. While physical co-localization of kinases with their substrate, often mediated by interactions between peptide binding domains and short linear interaction motifs, is a key component of substrate specificity, a growing body of work demonstrates that the kinase domain itself makes a significant contribution to substrate recognition. First, domain swapping experiments on receptor tyrosine kinases have shown that cells expressing chimeric receptors will develop a phosphorylation profile consistent with the substrate preference of the kinase domain rather than the base receptor. Complementing this work are studies on RET, CAMK2, c-Src, PKA, and ERK, which show that single-point mutations in kinase domains are sufficient to change their substrate preferences and that these changes can be pathogenic. Second, while kinase domains exhibit significant structural similarity, they display substantial variations in the charge and hydrophobicity of their surface residues. This observation, in combination with evolutionary analyses of tyrosine kinases, supports the hypothesis that they originated from a promiscuous common ancestor and evolved to develop distinct and diverse substrate preferences. Third, evolutionarily divergent residues have also been observed to span the kinase domain, suggesting that allostery plays an active role in substrate recognition.

Despite significant advances in profiling kinase-substrate interactions, there still lacks a comprehensive understanding of how kinase sequence and structure influence both kinase activity and substrate preferences. One particularly important knowledge gap is poor understanding of the impact of kinase variants on both of these properties. This is due to a shortage of data on the functional effect of missense mutations and limited structural data of kinase domains in complex with their substrates.

Existing methods for exploring kinase-substrate interactions have significant shortcomings when repurposed to study substrate specificity. For example, peptide positional scanning has generated consensus motifs for protein kinases at scale. Despite its utility, the lack of sequence-level resolution and the inability to assess kinase variants in parallel make it challenging to use this assay to study substrate specificity. Deep mutational scanning is another technology well-suited for understanding the function of kinase variants. In these experiments, the impact of thousands of mutations is measured in parallel by coupling protein activity to cell growth. Provided each variant is suitably barcoded, this coupling enables quantification by using next-generation sequencing to measure each variant's abundance (a surrogate of activity). Deep mutational scanning has been applied to various systems, including viral fitness, photosynthetic enzymes, G-protein-coupled receptors, protein-protein interactions, and kinases as discussed above. Recent work has demonstrated that performing deep mutational scans for protein activity and abundance jointly improves mechanistic understanding of the impact of mutations, as a protein's abundance is often a confounder of activity. Applying deep mutational scanning to study kinase substrate specificity is challenging because it requires coupling kinase substrate interactions to cell growth. Although studies have successfully performed mutational scans of ABL, MET, EGFR, Syk, ERK2, and c-Src kinases using various cell-based systems, every study is limited by a common set of challenges. Measurements in bacterial systems require the expression of functional kinases. Most studies do not distinguish between increased protein abundance and activity, although a recent study on c-Src in yeast has the potential to directly address this limitation by performing a separate mutational scan to directly measure the impact of variants on abundance. Cell line assays rely on specific cell lines that translate kinase activity into growth readouts, thereby limiting their generalizability. Last, and most critically, these prior studies measure overall kinase activity against broad substrate pools and are unable to reveal changes in substrate preferences among kinase variants.

The present disclosure bridges this gap by developing the phosphorylation protein complementation assay, or phospho-PCA. Phospho-PCA utilizes a growth-based biosensor, inspired by advances in fluorescent biosensors for protein kinases, to quantitatively couple kinase substrate interactions to the growth of budding yeast. The present disclosure demonstrates that phospho-PCA enables deep mutational scanning of kinase domains with the interaction strength for a specific substrate as the readout. The present disclosure further demonstrates that combining mutational scans with abundance-PCA and phospho-PCA with statistical modeling enables one to quantify the relative impact of mutations on kinase domain stability, kinase activity, and substrate preference. In some embodiments, mutational scans with abundance-PCA and phospho-PCA of Fyn, Lck, and c-Src kinases are performed to measure the impact of all single-point mutations for each kinase when paired with representative Fyn, Lck, and c-Src substrates. The results show that evolutionarily conserved sites are linked to significant decreases in abundance-PCA signal and that the combination of chemical properties and surface accessibility determines the impact of mutations on folding stability. Activity-biasing mutations were also identified using decoupled abundance-PCA and phospho-PCA scores. In some embodiments, decoupled scores are used to identify substrate-specificity-determining residues and to understand mechanistically how mutations at these residues impact substrate recognition when used in combination with computational modeling.

Disclosed herein includes a phosphorylation-protein complementation reporter system. In some embodiments, the phosphorylation-protein complementation reporter system comprises a first fusion protein comprising a kinase substrate linked to (i) a kinase domain and (ii) a first segment of a reporter molecule; and a second fusion protein comprising a phosphor-amino-acid binding domain (PAABD) linked to a second segment of the reporter molecule, wherein the first segment and the second segment of the reporter molecule are capable of complementing one another to form an active reporter molecule via the interaction between the PAABD and the kinase substrate upon phosphorylation by the kinase domain.

Disclosed herein also includes an abundance protein complementation reporter system. In some embodiments, the abundance protein complementation reporter system comprises a fusion protein comprising a kinase substrate linked to (i) a kinase domain and (ii) a first segment of a reporter molecule; and a second segment of the reporter molecule, wherein the first segment and the second segment of the reporter molecule are capable of complementing one another to form an active reporter molecule.

Disclosed herein also includes a method of detecting phosphorylation. The method can comprise introducing into a cell an expression vector comprising a polynucleotide encoding a phosphorylation-protein complementation reporter system described herein; culturing the cell in a selective culture medium under a condition for a time period, wherein the selective culture medium contains an inhibitor that inhibits cell proliferation in the absence of the active reporter molecule; and detecting a cell proliferation signal, wherein the cell proliferation signal indicates phosphorylation of the kinase substrate by the kinase domain.

Disclosed herein also includes a method of screening kinase variants. The method can comprise generating a kinase variant library comprising a plurality of expression vectors each comprising a polynucleotide encoding a phosphorylation protein complementation reporter system described herein, wherein the kinase domain comprises a variation and the polynucleotide comprises a variant barcode associated with the variation; introducing into a cell an expression vector of the plurality of expression vectors; culturing the cell in a selective culture medium under a condition for a time period, wherein the selective culture medium contains an inhibitor that inhibits cell proliferation in the absence of the active reporter molecule; extracting expression vectors from surviving cells; and determining the variation in the kinase domain by sequencing the extracted expression vectors.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

Unless specified otherwise, the left-hand end of any single-stranded polynucleotide sequence discussed herein is the 5′ end; the left-hand direction of double-stranded polynucleotide sequences is referred to as the 5′ direction.

The term “naturally occurring” as used herein refers to materials which are found in nature or a form of the materials that is found in nature.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990, the content of each of which is incorporated herein in its entirety.

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The phrase “substantially identical,” in the context of two nucleic acids or polypeptides (e.g., nucleic acids encoding a biosensor or a portion thereof, or the amino acid sequence of a biosensor or a portion thereof) refers to two or more sequences or subsequences that have at least about 60%, about 80%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, or over the full length of the two sequences to be compared.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein can be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

As used herein, the term “barcode” or “index” or “unique molecular identifier (UMI)” refers to a known nucleic acid sequence that allows some feature with which the barcode is associated to be identified. Typically, a barcode is unique to a particular feature in a pool of barcodes that differ from one another in sequence, and each of which is associated with a different feature. In embodiments, barcodes are about or at least about 3, 4, 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are 10⁻⁵⁰ nucleotides in length, such as 15-40 or 20-30 nucleotides in length. In a pool of different barcodes, barcodes may have the same or different lengths. In general, barcodes are of sufficient length and comprise sequences that are sufficiently different to allow the identification of associated features based on barcodes with which they are associated. In some embodiments, a barcode can be identified accurately after the mutation, insertion, or deletion of one or more nucleotides in the barcode sequence, such as the mutation, insertion, or deletion of 1, 2, 3, 4, 5, or more nucleotides. In some embodiments, each barcode in a plurality of barcodes differs from every other barcode in the plurality by at least three nucleotide positions, such as at least 3, 4, 5, 6, 7, 8, 9, 10, or more nucleotide positions.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In some embodiments, the solid substrate is gold, quartz, silica, plastic, glass, diamond, silver, metal, or polypropylene. In some embodiments, the solid substrate is porous.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “plasmid” refers to a nucleic acid that can be used to replicate recombinant DNA sequences within a host organism. The sequence can be a double stranded DNA.

As used herein, the term “element” refers to a separate or distinct part of something, for example, a nucleic acid sequence with a separate function within a longer nucleic acid sequence. The terms “transcription regulatory element” and “expression control element” are used to refer to nucleic acid molecules that can influence the expression (including at the transcription and/or translation level) of an operably linked coding sequence in a specific host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell. The promoter can be a specific promoter, e.g., cell type-specific and/or tissue-specific. The promoter can be constituent or inducible (e.g., by chemical agent, biological agent, temperature, and/or pH).

The term “transform” or “transfect” refers to the process of introducing an exogenous DNA inside a cell. The transformed DNA may or may not be integrated (covalently linked) into the genome of the cell. In prokaryotes, yeast, and mammalian cells for example, the transforming DNA may be maintained on an episomal element such as a plasmid. A “clone” is a population of cells derived from a single cell or common ancestor by mitosis. A “cell line” is a clone of a primary cell that is capable of stable growth in vitro for many generations. The nucleic acid may be in the form of naked DNA or RNA, associated with various proteins or the nucleic acid may be incorporated into a vector.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

### Phosphorylation- and Abundance-Protein Complementation Reporter Systems

Provided herein include the construction and applications of a phosphorylation protein complementation reporter system alone or in combination with an abundance protein complementation reporter system for high-throughput kinase variant screening. The related assays, methods, and expression vectors described herein can be used to detect phosphorylation and in deep mutational scanning. In some embodiments, the systems and related methods described herein utilize a growth-based biosensor to quantitatively couple kinase substrate interactions to the growth of budding yeast, thus enabling deep mutational scanning of kinase domains with the interaction strength for a specific substrate as the readout. When combined with statistical modelling, the methods and systems described herein can enable the quantification of the relative impact of mutations on kinase domain stability, kinase activity, and substrate preference.

In some embodiments, phosphor-protein complementation reporter system described herein comprises a first fusion protein comprising a kinase substrate linked to (i) a kinase domain and (ii) a first segment of a reporter molecule; and a second fusion protein comprising a phosphor-amino-acid binding domain (PAABD) linked to a second segment of the reporter molecule, wherein the first segment and the second segment of the reporter molecule are capable of complementing one another to form an active reporter molecule via the interaction between the PAABD and the kinase substrate upon phosphorylation by the kinase domain. In some embodiments, the kinase domain is capable of phosphorylating the kinase substrate.

The first fragment and the second fragment of the reporter molecule are complementing fragments. Complementing fragments refer to fragments of a polypeptide that are individually inactive, but binding of the complementing fragments restores activity of the reporter molecule. Therefore, if the first segment and the second segment of the reporter protein come into contact with one another, e.g., as a result of phosphorylation of the substrate by the kinase domain, they spontaneously self-complement to form an active reporter protein.

The kinase domain can be attached to one end of the kinase substrate (e.g., the N-terminal) and the first segment of the reporter molecule can be attached to the other end of the kinase substrate (e.g., the C-terminal). In some embodiments, a linker peptide is attached to the kinase domain and to the first segment of the reporter molecule to form a continuous structure in the following order: the kinase domain, the kinase substrate, and the first segment of the reporter molecule. In some embodiments, the linker peptide is flexible, allowing backbone flexibility and range of motion between the connected domains.

The term “kinase” as used herein generally refers to a type of enzymes that catalyse the transfer of phosphate groups from high energy, phosphate-donating molecules (e.g., ATP) to specific substrates, the process also known as phosphorylation. As a result, kinase produces a phosphorylated substrate. Kinases are classified into groups by the substrate they act upon: protein kinases, lipid kinases, and carbohydrate kinases. In some embodiments, the kinase used herein is a protein kinase. A protein kinase can be classified into various types based on their substrate specificity and regulatory mechanisms. Examples of protein kinases include serine/threonine kinases that phosphorylate serine and/or threonine residues, tyrosine kinases that phosphorylate tyrosine residues, dual-specificity kinases that can phosphorylate both serine/threonine and tyrosine residues. Other non-limiting examples of protein kinases include histidine kinases aspartate kinase, and glutamate kinase as will be understood by a person skilled in the art.

In some embodiments, the kinase domain used in the reporter system described herein is a serine-threonine kinase. Serine-threonine kinases (STKs) are a family of enzymes that catalyse the phosphorylation of serine and threonine amino acid residues, and particularly the OH group of serine or threonine, in proteins. Examples of STKs include, for example, protein kinase A (PKA), protein kinase C (PKC), mitogen-activated protein kinases (MAPKs), transforming growth factor-beta (TGF-β) receptor, and cyclin-dependent kinases (CDKs).

In some embodiments described herein, the kinase domain is a tyrosine kinase. A tyrosine kinase can be a receptor tyrosine kinase or a non-receptor tyrosine kinase. A receptor tyrosine kinase (RTK) refers to a family of transmembrane proteins that functions as cell surface receptors and typically consists of an extracellular domain that recognizes and binds specific ligands (e.g., growth factors or hormones), a transmembrane domain that anchors the receptor in the cell membrane, and an intracellular domain that contains a tyrosine kinase catalytic domain. Examples of RTKs include, but are not limited to, EGF receptor family, insulin receptor family, PDGF receptor family, VEGF receptor family, FGF receptor family, CCK receptor family, NGF receptor family, NGF receptor family, HGF receptor family, Eph recetpro family, AXL receptor family, TIE receptor family, RYK receptor family, DDR receptor family, RET receptor family, ROS receptor family, LTK receptor family, ROR receptor family, MuSK receptor family, LMR receptor family, and other identifiable to a person skilled in the art. Non-receptor tyrosine kinase (nRTK) refers to a class of tyrosine kinases that phosphorylates tyrosine residues on protein substrates without the need of a cell surface receptor. NRTKs typically consist of an intracellular catalytic domain responsible for phosphorylating tyrosine residue and a regulatory domain that controls kinase activity. Unlike RTKs, nRTKs lack receptor-like features such as an extracellular ligand-binding domain and a transmembrane-spanning region. Examples of a non-receptor tyrosine kinase include, but are not limited to, Src family, ABL family (Abl and Ab12), ACK family, CSK family, FAK family (e.g., FAK, Pyk2), FES family, FRK family, JAK family (e.g., JAK1, JAK2, JAK3, TYK2), SYK family, and TEC family (e.g., BMX, BTK, ITK).

In some embodiments, the kinase domain is a Src family kinase (SFK), including, for example, Src, Fgr, Fyn, Yes1, Blk, Hck, Lck, and Lyn, or a variant or a portion thereof. Across these SFKs, patient-documented variants include germline Src p.E527K causing thrombocytopenia with myelofibrosis and bleeding, somatic Src p.Q531* reported in advanced colorectal cancer, recurrent Fyn p.L174R/p.R176C/p. Y531H in peripheral T-cell lymphomas, and germline Lck p.L341P, p.P440S, p.C465R underlying combined T-cell immunodeficiency and intestinal inflammation. SFKs are a collection of non-receptor tyrosine kinases that play a role in various biological functions.

In some embodiments, the kinase domain used in the fusion protein described herein comprises c-Src, Lck, Lyn, or a portion or a variant thereof. These three kinases have 70-80% sequence similarity but play distinct roles in regulating differentiation, immune responses, and cell growth. Fyn has a broad tissue distribution and is involved in T-cell signaling and neuronal development; it is currently being investigated as a target for neurodegenerative diseases. Lck is expressed in T-cells and is a key component of the T-cell activation signaling cascade. c-Src is one of the most well-studied oncogenes; it is widely expressed and regulates cell adhesion, proliferation, and survival. The different roles these kinases have assumed are partly due to their varying substrate preferences.

The embodiments described in the Example section use Src family kinases such as Src, Lck, and Lyn as the kinase domain of a fusion protein to demonstrate the concept of phosphor-PCA and abundance-PCA as well as uses thereof in deep mutational scanning, but any kinase described herein will be suitable for use in the fusion proteins described herein.

For a given kinase, a person skilled in the art would be able to identify its substrate based on its phosphorylation-site sequence motif. Public databases (e.g., Kinase-Substrate Explorer (KiNet), Kinase-Logos) are available for kinase substrate recognition, providing information on the specific amino acid sequences (motifs) that protein kinases target for phosphorylation.

In some embodiments, a fusion protein described herein comprises a phosphor-amino acid binding domain connected to a fragment of a reporter molecule. The term “phosphor-amino acid binding domain” (PAABD) refers to a protein module that can recognize and bind to phosphorylated residues such as phosphorylated serine, threonine, and/or tyrosine on other proteins, thereby facilitating the formation of protein complexes and mediating protein-protein interactions. In some embodiments, the PAABD used herein can bind to a phosphotyrosine peptide, a phosphoserine peptide, and/or a phosphorthreonine peptide. Examples of PAABD include, but are not limited to, Src homology 2 (SH2) domain that can bind specifically to tyrosine-phosphorylated sequences, phosphotyrosine binding (PTB) domain that can bind to phosphotyrosine residues, 14-3-3 protein that can bind to phosphorylated serine and threonine residues, WD40 domain that can bind to phosphorylated serine or threonine residues, WW domains and forkhead-associated FHA domains that can recognize and bind phosphoserine and phosphorthreonine, respectively, and BRCA1 C-terminal (BRCT) domain that can act as a binding site for phosphorylated amino acids (pSer/pThr) and specific hydrophobic residues. In some embodiments described herein, the PAABD is a 14-3-3b, FHA, SH2, or WW domain.

A reporter molecule used in the phosphorylation-protein complementation reporter system described herein refers to any molecule that can exhibit a detectable and measurable activity, allowing it to monitor phosphorylation of a substrate by a kinase. In some embodiments, a reporter molecule can generate a fluorescent signal or is capable of binding small molecules that can be tagged with a fluorescent moiety to serve as a fluorescent probe. Reporters suitable for the phosphorylation-protein complementation reporter system described herein include, but are not limited to, dihydrofolate reductase (DHFR), green fluorescent protein (GFP) and two variants thereof (YFP and IFP), beta-lactamase, and Renilla luciferase (RLuc). For example, dihydrofolate reductase (DHFR) is capable of binding methotrexate with high affinity, and a methotrexate-fluorophore conjugate can serve as a quantitative fluorescent reagent for the measurement of the amount of DHFR within a cell.

In some embodiments, the reporter molecule is an enzyme. Interaction between the two fusion proteins can bring two fragments of the reporter enzyme in close proximity allowing them to form a functional enzyme whose activity as measured by cellular growth in selective conditions is proportional to the concentration of the protein complex, which in turn is proportional to the phosphorylation activity.

In some embodiments, the reporter molecule is an inhibitor-resistance conferring protein. An inhibitor-resistance conferring protein used herein refers to a protein, typically an enzyme, which can inactivate, metabolize, or neutralize a cell inhibitor, such as a drug or toxic chemical compound, thus enabling a cell to survive or grow in the presence of such inhibitor and conferring resistance to such inhibitor. In some embodiments, the inhibitor-resistance conferring protein is an antibiotic-resistance conferring protein.

The reporter molecule can be employed in a survival-selection system. Survival-selection assays are typically based on dominant or recessive selection, including selection based on the conferment of drug resistance or metabolic selection. Examples of reporter molecules that can be employed in a survival-selection system include, but are not limited to, beta-lactamase, adenosine deaminase, hygromycin-B-phosphotransferase, adenosine deaminase, L-histidinol: NAD+ oxidoreductase, xanthine-guanine phosphoribosyl transferase (XPRT), glutamine synthetase, asparagine synthetase, puromycin N-acetyltransferase, aminoglycoside phosphotransferase, bleomycin binding protein, cytosine methyltransferase, O6-alkylguanine alkyltransferase, glycinamide ribonucleotide (GAR) transformylase, glycinamide ribonucleotide synthetase, phosphoribosyl-aminoimidazole synthetase, formylglycinamide ribotide amidotransferase, phosphoribosyl-aminoimidazole carboxamide formyltransferase, fatty acid synthease, IMP dehydrogenase as well as other proteins that can enable cell survival or growth under specific conditions. These reporter molecules can be dissected into fragments, a first and second fragment, corresponding to an N-terminal fragment and a C-terminal fragment, and used as a reporter molecule in the reporter systems described herein, such that a cell survives under certain conditions if the first and second fragments complement with one another and form a functional protein. It will be apparent to one skilled in the art that a variety of measures of cell survival or cell growth can be employed for detection, including cell number, DNA content or protein content, optical density, and cell staining.

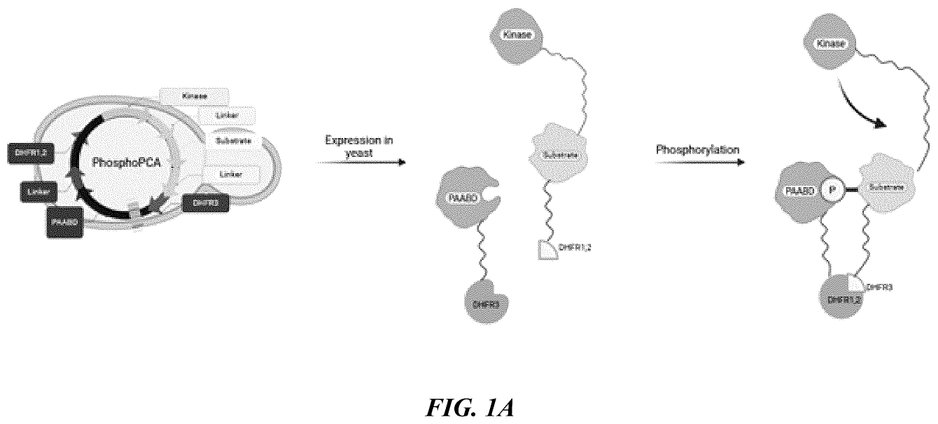

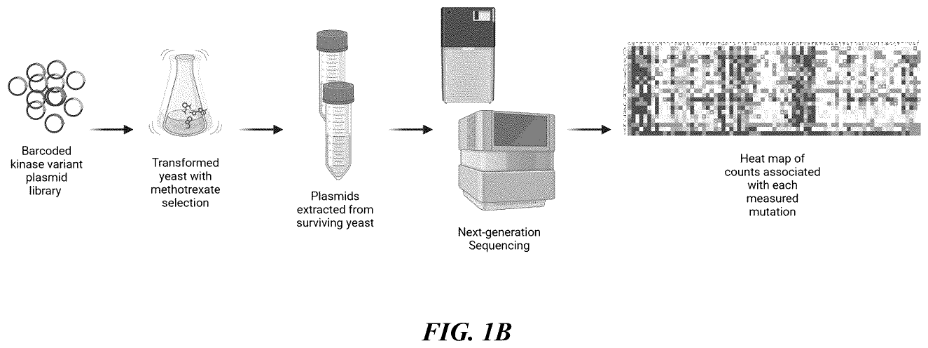

Accordingly, in some embodiments, the reporter molecule can be used in conjunction with a cell growth-based assay to generate a detectable cell growth profile (e.g., growth curves or OD600 in liquid culture). For example, phosphor-PCA can use a growth-based biosensor to couple kinase-substrate interaction to the growth of budding yeast cells (see, for example, FIGS. 1A-1B).

In some embodiments, the reporter protein is dihydrofolate reductase (DHFR), an enzyme involved in folate metabolism. The phosphor-protein complementation reporter system described herein can comprise two complementary fragments of the DHFR that are fused to two fusion proteins. Accordingly, the phosphor-protein complementation reporter system can comprise a first fusion protein comprising a kinase substrate linked to (i) a kinase domain and (ii) a first DHFR segment; and a second fusion protein comprising a phosphor-amino-acid binding domain (PAABD) linked to a second DHFR segment. In some embodiments, the kinase domain is capable of phosphorylating the kinase substrate. Upon phosphorylation of the substrate by the kinase domain, the first fusion protein and the second fusion protein interact with each other via the interaction between the PAABD and the phosphorylated substrate, bringing the two DHFR fragments into proximity, which in turn leads to the reconstitution of the enzyme activity. The phosphor-protein complementation reporter system using a DHFR reporter molecule can be a survival-selection assay in which yeast is inhibited by methotrexate, thus preventing nucleotide synthesis and causing arrest of cell division. Reconstitution of an active DHFR restores nucleotide synthesis, allowing cell proliferation. In some embodiments, the kinase domain is not capable of phosphorylating the kinase substrate due to, for example, variations in the kinase domain and/or kinase-substrate mismatch. In such scenarios, the first fusion protein and the second fusion protein will not interact with each other, and accordingly the reconstitution of the enzyme activity will not occur, leading to cell death.

The signal obtained using this assay (i.e., growth curves or OD600 in liquid culture) correlates to the amount of protein complexes formed by the two fusion proteins (also the kinase-substrate strength) as a result of phosphorylation of the substrate. Therefore, substrate phosphorylation by the kinase domain translates into growth of yeast strains in presence of the drug methotrexate. In some embodiments, the DHFR can include conservatively modified variants as long as the conservatively modified variant retains the characteristics of the enzyme. In some embodiments, the DHFR is a methotrexate-resistant DHFR. In some embodiments, the DHFR is a mutant (e.g., L22F and F31S) and is less sensitive to methotrexate than wild-type Saccharomyces cerevisiae DHFR.

The phosphorylation-protein complementation reporter system described herein can be used in conjunction with an abundance protein complementation reporter system. An abundance protein complementation reporter system can comprise, in some embodiments, a fusion protein comprising a kinase substrate linked to (i) a kinase domain and (ii) a first segment of a reporter molecule; and a second segment of the reporter molecule, wherein the first segment and the second segment of the reporter molecule are capable of complementing one another to form an active reporter molecule. The abundance protein complementation reporter system differs from the phosphorylation-protein complementation reporter system in that the former does not contain the PAABD. Functional reporter molecule can be reconstituted by random encounters between the fusion protein and the second fragment of the reporter molecule. If the first segment of the reporter molecule and the second segment of the reporter molecule come into contact with one another, they spontaneously self-complement to form a functional reporter protein. The second segment of the reporter molecule can be expressed at the same level as or a different level from the fusion protein. In some embodiments, the second segment of the reporter molecule is expressed at a higher level than the fusion protein. In some embodiments, the fusion protein and the second segment of the abundance protein complementation reporter system are expressed at the same level as the first fusion protein and the second fusion protein of the phosphorylation-protein complementation reporter system, respectively, when used in conjunction.

The kinase domain may or may not interact or phosphorylate the kinase substrate. The kinase domain, the substrate and/or the reporter molecule can be the same as those in the phosphorylation-protein complementation reporter system described above when used together. For example, the kinase domain can a tyrosine kinase or a portion thereof, optionally the kinase domain is a Src family kinase (SFK) or a portion thereof. In some embodiments, the kinase domain is a serine/threonine kinase. In some embodiments, the kinase domain is Fyn, Lck, or c-Src. The reporter molecule can be any of the reporter molecule described herein, including for example an inhibitor-resistance conferring protein such as DHFR.

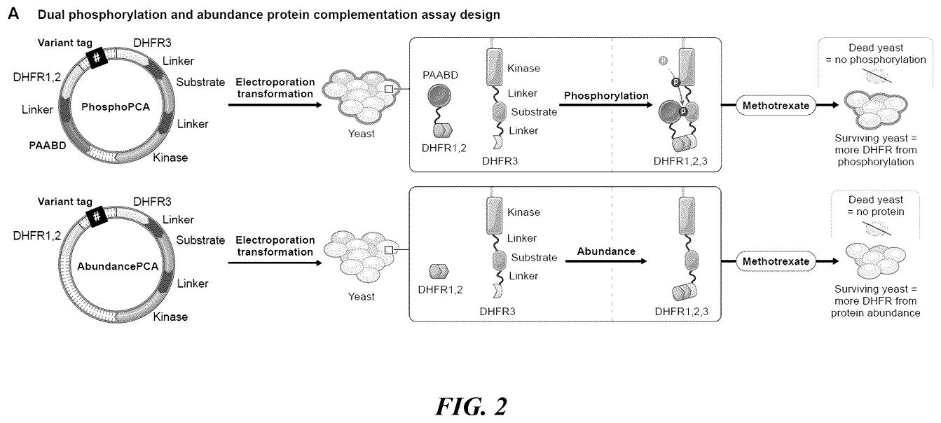

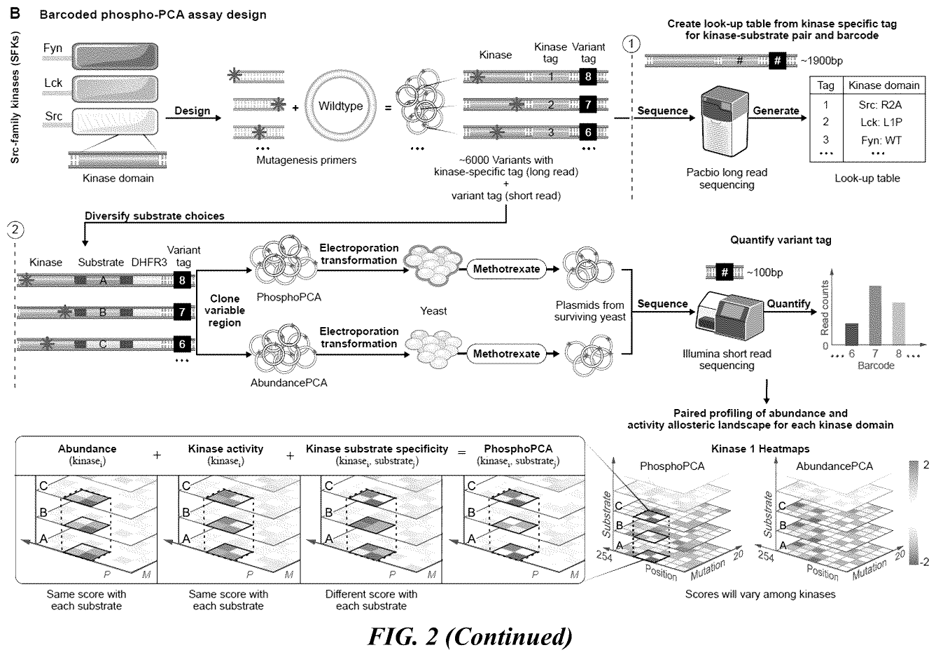

Exemplified embodiments described herein (see, for example, Example 2 and FIG. 2, panel A) employ a phosphor-protein complementation reporter system and/or an abundance protein complementation reporter system using DHFR as the reporter molecule in both reporter systems, but it will be understood by a person skilled in the art that the present disclosure is not limited to any particular reporter molecule presented herein. Any reporter molecule capable of generating a detectable signal (e.g., in selective conditions and measurable by cellular growth) can be utilized to create a phosphorylation-protein complementation reporter system and abundance protein complementation reporter systems as well as employed in the phosphor-complementation assay and abundance complementation assay described herein.

Provided herein also includes an expression vector comprising a polynucleotide encoding the phosphorylation-protein complementation reporter system and an expression vector comprising a polynucleotide encoding the abundance protein complementation reporter system described herein. The expression vector can be any construct that facilitates the delivery of the nucleic acid to the target cell and/or expression of the nucleic acid within the cell. Expression vectors can comprise plasmid DNA, viral vectors, or non-viral vectors, among others known to those skilled in the art, comprising appropriate regulatory elements such as promoters, enhancers, and post-transcriptional and post-translational regulatory sequences that are compatible with the bacteria or yeast heterologously expressing the reporter systems, as would be understood by a skilled person. In some embodiments, the expression vector is a plasmid DNA.

Polynucleotides encoding the first fusion protein and the second fusion protein of the phosphorylation-protein complementation reporter system can be on a single plasmid. Similarly, polynucleotides encoding the fusion protein and the second segment of the reporter molecule of the abundance protein complementation reporter system can be on a single plasmid.

In some embodiments, the plasmid further comprises a unique molecule identifier, also known as a barcode, which is a known nucleic acid sequence that allows some feature with which the barcode is associated to be identified. In some embodiments the feature with which the barcode is associated to be identified is a variation in the fusion protein such as a variation in the kinase domain. For example, the kinase domain of the reporter system may comprise a variation with respect to its wild type sequence, including for example one or more substitution, deletion, and/or insertion. A barcode can be designed to associate with features of the variation including the position of the variation, the mutated amino acid as well as the wild-type amino acid. In some embodiment, the plasmid can further comprise a second barcode. The second barcode can be associated with the kinase substrate domain in the fusion protein or a variation therein.

In some embodiments, barcodes are about, at least about, or at most about 3, 4, 5, 6, 7, 8, 9, 10, 15, 20, 25, 30, 40, 50, or more nucleotides in length. In some embodiments, barcodes are shorter than 30, 20, 15, 10, 9, 8, 7, 6, or 5 nucleotides in length. In some embodiments, barcodes are 10-50 nucleotides in length, such as 15-40 or 20-30 nucleotides in length. Barcodes may have the same or different lengths. In general, barcodes are of sufficient length and comprise sequences that are sufficiently different to allow the identification of associated features based on barcodes with which they are associated.

The barcoded phosphor-protein complementation reporter system alone or in combination with barcoded abundance-protein complementation reporter system, when coupled with next-generation sequencing, can measure each variant's kinase-substrate activity in parallel, therefore enabling the feasibility of applying deep mutational scans to study kinase substrate specificity in high throughput and with enhanced accuracy.

Provided herein also include protein complementation assays including a phosphorylation-protein complementation assay (phosphor-PCA) and an abundance-protein complementation assay (abundance-PCA). A phosphor-PCA can be used alone or in combination with an abundance-PCA for the detection of kinase phosphorylation. In some embodiments, a phosphor-PCA for the detection of phosphorylation comprises a phosphorylation-protein complementation reporter system described herein, wherein the phosphorylation is detected by the reconstitution of the active reporter molecule.

Provided herein also include a method for constructing a phosphor-PCA. In some embodiments, the method comprises providing a phosphorylation-protein complementation reporter system herein described, and measuring the activity of the reporter molecule. In some embodiments, the phosphor-PCA can be used in conjunction with a cell growth assay, and the activity of the reporter molecule can be quantified as a cell proliferation signal such as a cell growth rate. Cell proliferation signals can be detected and quantified with a variety of automated and/or high-throughput instrumentation systems such as automated cell-based imaging systems that provide spatial resolution of the signal. The assays and methods described herein can accelerate kinase-substrate interaction profiling for a large number of candidates that may be difficult to screen by conventional methods.

### Phosphorylation Detection and Variant Screening

Provided herein include phosphorylation- and abundance-protein complementation report systems and uses thereof in related kinase protein complementation assays to detect phosphorylation. The methods and systems can be used to investigate kinase-substrate interaction and to identify mutations that impact kinase activity and substrate specificity through allostery. In some embodiments, the methods and systems described herein can quantitatively couple kinase substrate interactions to cell growth, thus enabling deep mutational scanning of kinase variants with the interaction strength for a specific substrate as the readout. In some embodiments, when combining phosphorylation-protein complementation assay (phosphor-PCA) with abundance-protein complementation assay (abundance-PCA), the methods can enable the quantification of the relative impact of mutations on kinase domain stability, kinase activity and substrate preference. The methods and assays described herein can be used in high-throughput screening of kinase variants to investigate how mutations impact their enzymatic activity and substrate preferences.

Provided herein includes a method of detecting phosphorylation. In some embodiments, the method comprises introducing into a cell an expression vector comprising a polynucleotide encoding a phosphorylation-protein complementation reporter system described herein; culturing the cell in a selective culture medium under a condition for a time period, wherein the selective culture medium contains an inhibitor that inhibits cell proliferation in the absence of the active reporter molecule; and detecting a cell proliferation signal, wherein the cell proliferation signal indicates phosphorylation of the kinase substrate by the kinase domain.

Introducing one or more expression vectors into a cell can be performed by methods identifiable by a skilled person upon reading of the present disclosure. Methods of transforming of prokaryotic cells and eukaryotic cells such as electroporation, heat-shock transformation and others are well known in the art and described herein (e.g., see Examples). In some embodiments, the cell used herein for the transformation is a yeast cell.

The phosphorylation-protein complementation reporter system described herein can be used in conjunction with a survival-selection system such that a cell only survives in a selective culture media if the first segment and the second segment of the reporter molecule complement with one another to form an active reporter molecule as a result of substrate phosphorylation.

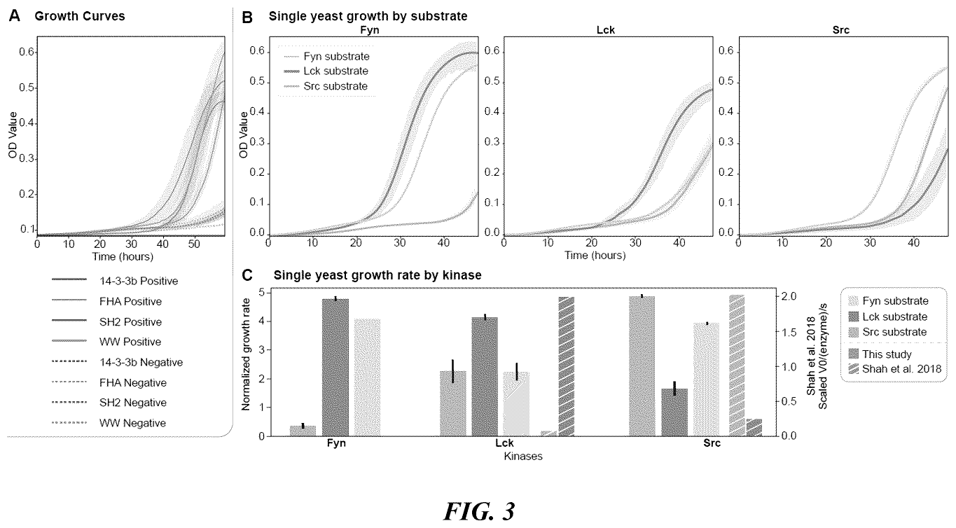

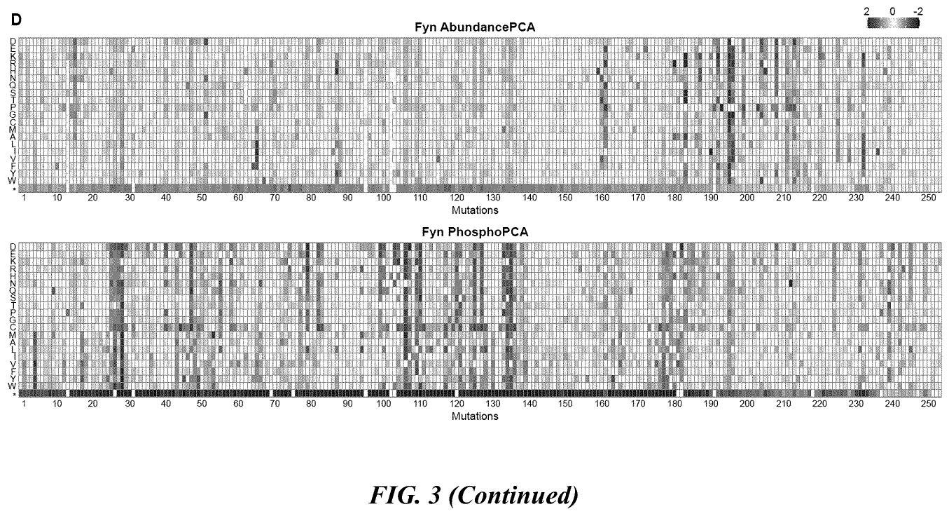

Accordingly, the method can further comprise culturing the cell containing the expression vector in a selective culture medium under a condition for a time period allowing the cell to proliferate. The selective culture medium contains substances needed for cell growth and proliferation as well as an inhibitor that inhibits cell proliferation in the absence of the active reporter molecule. In some embodiments, the active reporter molecule is an inhibitor-resistance conferring protein, such as a DHFR, and the inhibitor can be methotrexate. If a kinase-substrate interaction occurs, the substrate is phosphorylated, leading to the binding of the PAABD to the phosphorylated substrate, which in turn reconstitute the reporter molecule (e.g., DHFR), conferring resistance to the inhibitor (e.g., methotrexate). Cell proliferation can be detected by measuring a cell proliferation signal such as optical density (OD600) over time, CFU counts, flow cytometry, plasmid contents, as well as other standard techniques identifiable to a person skilled in the art. In some embodiments, detecting a cell proliferation signal can comprise generating a cell growth profile and/or a cell growth rate which can be extracted during the exponential phase of cell growth. The cell proliferation signal can be used to quantify the strength of kinase-substrate interactions during cell growth. The cell proliferation signal detected above can also be referred to as a phosphor-PCA signal or a phosphor-PCA score. An absence of a phosphor-PCA signal or a phosphor-PCA score below a threshold value indicates that no phosphorylation occurs. An exemplary embodiment of a phosphor-PCA is demonstrated in FIG. 2, panel A and FIG. 3, panels A-C, showing biosensors containing interacting kinase-substrate pairs resist selection, while biosensors with non-interacting pairs display significantly reduced growth.

In some embodiments, the method further comprises introducing into a cell an expression vector encoding an abundance-protein complementation reporter system described herein, culturing the cell in a selective culture medium under a condition for a time period, wherein the selective culture medium contains an inhibitor that inhibits cell proliferation in the absence of the active reporter molecule; and detecting an abundance cell signal (or an abundance-PCA signal). The cell can be a yeast cell. In some embodiments, detecting the abundance cell signal and detecting the cell proliferation signal are performed in parallel. A non-limiting, exemplary embodiment of a dual phosphorylation and abundance protein complementation assay is illustrated in FIG. 2, panel A.

As described above, an abundance protein complementation reporter system can comprise, in some embodiments, a fusion protein comprising a kinase substrate linked to (i) a kinase domain and (ii) a first segment of a reporter molecule; and a second segment of the reporter molecule, wherein the first segment and the second segment of the reporter molecule are capable of complementing one another to form an active reporter molecule. The abundance protein complementation reporter system differs from the phosphorylation-protein complementation reporter system used above in that the former does not contain the PAABD. Functional reporter molecule can be reconstituted by random encounters between the fusion protein and the second fragment of the reporter molecule.

As will be understood by a person skilled in the art, the phosphorylation score measured by the cell proliferation signal depends on protein folding stability, the probability the kinase is in an active state, and the specificity of the kinase-substrate interaction. Since protein misfolding can reduce kinase abundance, which in turn decreases phosphorylation, folding stability acts as a confounder that needs to be separated from the other two effects. The abundance score measured by the abundance cell signal quantifies how a mutation can alter the abundance of the kinase itself (e.g., the concentration of stable kinase). The abundance cell signal can be detected in the same way the cell proliferation signal is detected, such as from measuring optical density (OD600) over time and generating a cell growth curve and/or a cell growth rate.

The method further comprises applying a hierarchical Bayesian regression model to the cell proliferation signal and the abundance cell signal to decompose the cell proliferation signal into a sum of scores each representing kinase stability, kinase activity or substrate specificity. The hierarchical Bayesian regression model used here can decouple kinase-substrate interaction measured by phosphor-PCA from the kinase folding and stability measured by the abundance-PCA. In an exemplary embodiments, the observed phosphor-PCA signal (i.e., the cell proliferation signal detected from the phosphor-PCA) is decomposed into three distinct components:

$$phospho - P C {A}_{i j k l} = {a}_{i j} \times {Abundance}_{i k l} + {b}_{i j} + {Activity}_{i k l} + Substrate {specificity}_{ijkl} ,$$

In some embodiments, the cell proliferation signal is decomposed into a linear sum of (a) the abundance cell signal multiplied by an abundance effect slope parameter; (b) an intercept representing the baseline phosphorylation activity independent of abundance effects; (c) a score representing kinase activity effect; and (d) a score representing substrate specificity effect. The abundance effect slope parameter captures how abundance affects phosphor-PCA scores for each specific kinase-substrate pair). (a) and (b) represent kinase-substrate experiment effects, (c) represents kinase activity effects by grouping mutations according to kinase identity, mutation position, and amino acid change, and (d) represents kinase-substrate specificity effects captured by four factors: kinase identity (i), substrate (j), mutation position (k), and amino acid change (l). The model can statistically separate true changes in enzymatic activity from abundance-driven changes, enabling more precise inference of the functional impact of mutations. Detailed description of decoupling abundance- and phosphor-PCA data through hierarchical Bayesian modeling is provided in Example 1.

In some embodiments, the method can further comprise identifying one or more mutations, based on the decomposition of the cell proliferation signal. The one or more mutations can (i) negatively impact kinase catalytic activity and negatively impact kinase stability, (ii) negatively impact kinase catalytic activity and have minimal or no effect on abundance, and/or (iii) positively impact kinase catalytic activity.

In some embodiments, the method can further comprise identifying, based on cell proliferation signal and the abundance cell signal, one or more potential allosteric sites coupled to substrate specificity and/or kinase activity. Allosteric sites in the context of a kinase refer to sites that differ from the active site of the enzyme but control the phosphorylation activity through allostery. While orthosteric sites are conserved across families and super-families of proteins, allosteric sites are not conserved (and have unique structure and properties) and therefore offer the opportunity of targeting specific proteins. Targeting allosteric sites that communicate with the active site or substrate interface can induce a conformational change in the kinase, which can generate potent and specific molecules with modulated kinase activity. The method described herein can be combined with computational modeling to identify variations/residues that control kinase catalytic activity as well as variations/residues that determine substrate specificity. In some embodiments, molecular dynamics of selected mutants (e.g., specificity-determining mutants) can be performed to investigate the allosteric networks and to identify residues governing substrate specificity.

Provided herein also include methods of screening kinase variants. In some embodiments, a method of screening kinase variants comprises generating a kinase variant library comprising a plurality of expression vectors each comprising a polynucleotide encoding a phosphorylation protein complementation reporter system described herein, wherein the kinase domain comprises a variation and the polynucleotide comprises a variant barcode associated with the variation; introducing into a cell an expression vector of the plurality of expression vectors; culturing the cell in a selective culture medium under a condition for a time period, wherein the selective culture medium contains an inhibitor that inhibits cell proliferation in the absence of the active reporter molecule; extracting expression vectors from surviving cells; and determining the variation in the kinase domain by sequencing the extracted expression vectors. In some embodiments, the expression vectors extracted from surviving cells contain a kinase variant capable of phosphorylating the kinase substrate. In some embodiments, the cells are yeast cells.

In some embodiments, the method further comprises generating a kinase variant library comprising a plurality of expression vectors each comprising a polynucleotide encoding an abundance-protein complementation reporter system described herein, wherein the kinase domain comprises a variation and the polynucleotide comprises a variant barcode associated with the variation; introducing into a cell an expression vector of the plurality of expression vectors; culturing the cell in a selective culture medium under a condition for a time period, wherein the selective culture medium contains an inhibitor that inhibits cell proliferation in the absence of the active reporter molecule; extracting expression vectors from surviving cells; and determining the variation in the kinase domain by sequencing the extracted expression vectors. As described above, an abundance protein complementation reporter system can be constructed from the phosphorylation-protein complementation reporter system by removing the PAABD. A non-limiting, exemplary embodiment of barcoded phosphor-PCA assay and barcoded abundance-PCA assay is illustrated in FIG. 2, panel B.

In some embodiments, a library of kinase domain variants comprises a plurality of expression vectors each comprising a polynucleotide encoding for a phosphorylation protein complementation reporter system that contains a variation in the kinase domain. The variation in the kinase domain can be a substitution, insertion, and/or deletion relative to the wild type sequence of the kinase domain. In some embodiments, a kinase variant contains a single amino acid substitution in the kinase domain relative to a wild type kinase.

The number of expression vectors in the kinase variant library can vary in different embodiments. In some embodiments, the kinase variant library can comprise at least, at least about, at most, or at most about 10, 100, 500, 1000, 2000, 3000, 4000, 5000, 6000, 7000, 8000, 9,000, 10,000, 20,000, 30,000, 40,000, 50,000, or a number or a range between any two of these values, vectors. Accordingly, the method described herein can be configured to screen a large number of kinase variants in parallel, thus providing cost savings, time saving, and uniformity of conditions.

In some embodiments, the methods and assays described herein comprise constructing a kinase variant library for deep mutational scanning. Deep mutational scanning is a high-throughput method utilizing a library of mutants covering most (or all) possible mutations in a protein. Such libraries contain thousands of unique sequences, which can be used simultaneously in functional screening experiments using in vitro selection strategies. A kinase variant library can be generated using any suitable method known in the art. In some embodiments, the kinase variant library is generated using site-directed saturation mutagenesis which introduces all possible mutations at one or more specific residues in the kinase domain. Degenerate primers (e.g., NNK or NNS codons) can be designed to target specific amino acids, followed by PCR-based mutagenesis to generate a screening plasmid library which can then be cloned into a vector for expression. For example, a comprehensive saturation mutagenesis can be performed using a one-pot method with NNK primer sets designed for each kinase domain as will be understood by a person skilled in the art. In some embodiments, error-prone PCR can be used to introduce random mutations across the kinase domain. The kinase variant library encoding the abundance protein complementation reporter systems can be constructed in the same way as described above with respect to the phosphorylation-protein complementation reporter system.

The polynucleotides in the kinase variant library can contain variations with respect to a same wild type kinase (e.g., Fyn). Alternatively, the polynucleotides in a kinase variant library can contain variations with respect to different wild type kinases such as variations relative to Fyn, Lck, c-Src, and others. In such scenarios, the vector can further comprise a kinase barcode allowing for the identification of the wild type kinase a variation is based upon. In some embodiments, the kinase variant library comprises polynucleotides encoding different kinase substrates. For example, a complete 3×3 kinase-substrate panel can be generated for wild type kinases Fyn, Lck, and c-Src. Each kinase variant (e.g., Fyn, Lck, or c-Src variants) can be assembled with each substrate (Fyn substrate, Lck substrate, or c-Src substrate), yielding nine phosphor-PCA libraries. Matched abundance-PCA libraries can be constructed in the same manner. The cloned kinase variant library can be transformed into a cell such as a budding yeast cell, and the activity of each variant for phosphor-PCA (as well as abundance-PCA) can be measured by quantifying cell growth in the presence of the inhibitor (e.g., methotrexate when the reporter molecule is DHFR) with sequencing (e.g., short-end sequencing).

Each expression vector of the plurality of expression vectors comprises a polynucleotide encoding for the phosphorylation protein complementation reporter system or a polynucleotide encoding for the abundance protein complementation reporter system described herein. For example, the polynucleotide can comprise DNA sequence for a first segment of the reporter molecule, a second segment of the reporter molecule, one or more linker, a substrate, a kinase variant, and a PPBD (in the phosphorylation-protein complementation reporter system), The kinase variant is a variant of a wild type kinase domain containing one or more mutation, deletion, and/or insertion in the sequence relative to its wild type sequence. The polynucleotide further comprises a variant barcode associated with the variation. Therefore, identity of the variation can be determined based on the associated variant barcode using, for example, a lookup table that links kinase variants to variant barcode sequences. Accordingly, in some embodiments, determining the variation in the kinase domain comprises sequencing the variant barcode, optionally using short-read sequencing, and determining the variation in the kinase domain using a lookup table. In the lookup table, the wild-type amino acid, the position of the variation, and the mutated amino acid describe each variant, and each variant correspond to a unique variant barcode sequence. Long read sequencing of the expression vector can be performed to construct a barcode-variant lookup table. In some embodiments, the method employs Bayesian statistics to integrate sequence information across all reads with a common barcode to estimate the consensus variant sequence for each barcode. Barcode sequences are matched to the lookup table using exact sequence identity.

In some embodiments, the method further comprises determining a relative abundance for each variant barcode. The barcode region can be amplified first and then sequenced to obtain sequencing reads. The relative abundance of each barcode in the pooled library can be determined based on the number of sequencing reads corresponding to each barcode. The sequencing can be performed using any next-generation sequencing platforms as will be understood by a person skilled in the art. In some embodiments, the sequencing is performed using an Illumina® sequencing platform. The method can further comprise normalizing the abundance of each barcode to the total number of sequencing reads obtained in the sample. In some embodiments, only barcodes with a read count above a predefined threshold are retained for downstream analysis.

With the phosphorylation-protein complementation reporter system, the relative abundance value obtained for each kinase variant is indicative of the concentration of formed protein complexes, which in turn is proportional to the phosphorylation activity of the variant (e.g., kinase-substrate interaction strength). With the abundance-protein complementation reporter system, the relative abundance value is indicative of the overall protein stability. The relative abundance values obtained from the two kinase complementation reporter systems can be used as a phosphor-PCA signal representing kinase-substrate interaction and an abundance-PCA signal representing kinase stability. The method further comprises assigning a phosphor-PCA signal and/or an abundance-PCA signal to each variation in the kinase variant library, for example, based on the variant barcode sequence.

Similar to the cell proliferation/abundance signals obtained from the standard phosphor-PCA and abundance-PCA assays described above, the relative abundance values obtained from barcoded phosphor-PCA can also be decoupled from the relative abundance values obtained from barcoded abundance-PCA and decomposed into three components: a score representing kinase abundance, a score representing kinase activity effect by the mutations, and a score representing kinase-substrate specificity (see for example, FIG. 2 panel B).

The assays and methods described herein can generate comprehensive maps of how sequence variation impacts kinase function including kinase activity and kinase-substrate specificity systematically in high throughput and with a resolution that would be difficult to achieve with conventional biochemical approaches.

### EXAMPLES

Some aspects of the embodiments discussed above are disclosed in further detail in the following examples, which are not in any way intended to limit the scope of the present disclosure.

### Example 1

### Materials and Methods

This example presents general materials and methods used in Example 2 below.

### Single Yeast Plasmid Construction

Plasmid construction began with two protein complementation assay (PCA) backbones: pGJJ001 (binding-PCA containing DHFR fragments under dual CYC promoters) and pGJJ045 (abundance-PCA containing DHFR fragments under CYC and GPD promoters). An SH2 domain was first introduced into each backbone using BamHI/SpeI restriction sites, generating pCY001 (CYC/CYC configuration) and pCY002 (GPD/CYC configuration). Active and catalytically inactive Fyn kinase domains were subsequently cloned via NheI/HindIII sites, yielding pCY003-004 from the pCY001 backbone and pCY005-006 from the pCY002 backbone. Using pCY005 as the template, a comprehensive panel of kinase GSlinker-substrate fusion constructs was assembled through identical NheI/HindIII cloning strategies. This approach generated plasmids pKMS003-008 and pKMS012-016, encompassing all combinations of Fyn, Src, and Lck catalytic domains paired with their respective substrate sequences (exemplified by fyn-gs-srcsub, src-gs-lcksub, lck-gs-fynsub). Control plasmids containing catalytically dead kinase domains (pCY006, pKMS015/016) were constructed in parallel to establish baseline measurements.

### Yeast Transformation

Growth-based biosensors were introduced into yeast through transformation with the corresponding plasmid. Yeast cultures were inoculated in 6 mL YPD medium and grown overnight at 30° C. Cultures were diluted to OD600=0.1 in 180 mL YPD and grown to OD600=0.5-0.6. For lithium acetate/polyethylene glycol transformation, approximately 45 mL of cells were harvested, washed twice in 100 mM lithium acetate, and aliquoted into 1.5 mL microcentrifuge tubes. Salmon sperm DNA was prepared by boiling for 5 minutes followed by ice incubation. Transformation mixtures were prepared by sequential addition to cell pellets: 240 L 50% PEG-3350, 36 L 1M lithium acetate, 10 L salmon sperm DNA (10 mg/mL), and 74 L plasmid DNA (1-5 g in a total volume of 360 L). After vortexing, samples were incubated at 30° C. for 30 minutes, heat-shocked at 42° C. for 30 minutes, pelleted, resuspended in 150 L water, and plated on SC-URA selective medium for 2-4 days.

### Quantitative Growth-Based Kinase-Substrate Interaction Assays

Transformed colonies were picked into 96-deep-well plates containing 500 L SC-URA/MET/ADE medium per well and grown overnight at 30° C. Cell density was measured by transferring 100 L culture plus 100 L fresh medium to flat-bottom 96-well plates. Remaining cultures were preserved as glycerol stocks (30% final glycerol concentration) and diluted to OD600=0.1. For kinase activity measurements, 5 L diluted cells were added to 95 L competition medium (1.053× SC-URA/MET/ADE supplemented with 200 g/mL methotrexate) to achieve a starting density of OD600=0.005. Growth was monitored for 60 hours at 30° C. using a Tecan plate reader with OD600 measurements every 15 minutes.

Time-course data were smoothed using a centered moving average (25-point window). Following exclusion of an initial burn-in period (100 smoothed points), exponential growth phases were identified by computing the numerical derivative of smoothed optical density with respect to time. Time points exceeding a dynamic threshold (T, initially 5×10−3, iteratively reduced by a factor of 1.25) were selected until at least 25 points were retained. When insufficient points met the criteria or the final OD600 was below 0.05, the entire post-burn-in trace was analyzed. Growth rates were calculated as the slope across selected exponential-phase windows. To allow cross-kinase comparisons, growth rates were normalized by the z-score within each kinase and then scaled to the range [0,2]. Condition-level summaries represent median normalized replicate values with variability expressed as standard deviation unless otherwise specified.

### Construction of Deep Mutational Scanning Libraries

To create barcoded biosensor libraries, we first constructed modular screening plasmids from the abundance-PCA and phospho-PCA plasmids by introducing a BamHI site upstream of the (GGSGG)4 linker using KpnI and NheI restriction sites. These modifications generated matched kinase-substrate PCA constructs: pDMS001 (abundance-PCA; Fyn kinase with Fyn substrate) and pDMS002-004 (phospho-PCA; Fyn, Lck, and Src kinases with their respective substrates). Kinase open reading frames were subcloned into the mutagenesis vector pGJJ055 via BamHI/HindIII to create kinase-specific templates pDMS005 (Fyn), pDMS006 (Lck), and pDMS007 (Src). Comprehensive saturation mutagenesis was performed using the one-pot method with NNK primer sets designed for each kinase domain. Following electroporation into TOP10 electrocompetent cells and overnight growth, mutant libraries were harvested by plate scraping and plasmid DNA prepared by midiprep. BamHI/HindIII kinase fragments were shuttled into pDMS002 to generate phospho-PCA libraries containing the Fyn substrate. Libraries were linearized at the barcode landing pad (AvrII/BsiWI) and barcoded using IDT oligonucleotide cassettes with NEBuilder Assembly. Each barcoded pool was bottlenecked to approximately 3% of transformants to maintain library diversity while ensuring adequate representation. To generate the complete 3×3 kinase-substrate panel, substrate cassettes were exchanged using AvrII/BamHI digestion and ligation with fragments from pDMS003 and pDMS004 to assemble barcode-[Fyn/Lck/Src] Mut-[fynsub/lcksub/srcsub], yielding nine phospho-PCA libraries. Matched abundance-PCA libraries were constructed by ligating kinase-substrate-barcode cassettes into pGJJ045 via BsiWI/HindIII to generate barcode-[Fyn/Lck/Src] Mut-[fynsub/lcksub/srcsub]-noSH2.

Selection of Barcoded Biosensor Libraries with Methotrexate

BY4742 yeast cells were prepared for electroporation by growth to mid-log phase (OD600≈1.6), followed by washing with ice-cold water and electroporation buffer. Cells were incubated in 0.1 M LiAc/10 mM DTT for 20 minutes at 30° C. before final preparation. Libraries (0.3 g) were electroporated with salmon sperm DNA (50 g) using standard parameters (2.5 kV, 25 F, 200). Transformed cells were rescued in sorbitol/YPD medium and expanded in SC-URA medium for 48 hours. Following initial selection on uracil-deficient media, input samples were grown for approximately 4 generations in SC-URA/MET/ADE medium. Competition experiments were initiated by transferring cells into medium containing 200 g/mL methotrexate at OD600=0.05 and growing for approximately 5 generations. For each library, 12-16 electroporation reactions were pooled across two biological replicates, requiring 1.35 L of medium per replicate. Cell pellets were harvested by centrifugation and stored at −20° C. for subsequent plasmid extraction using ZymoPrep II columns.

### Library Preparation for Next Generation Sequencing

Barcode regions were amplified using a two-step PCR protocol. In PCR1, eight parallel 50 L reactions per library were performed using KAPA HiFi polymerase with barcode-flanking primers for 15 cycles at 65° C. annealing temperature. Products were pooled and purified using DNA Clean Concentrator-5 columns. PCR2 appended Illumina adapter sequences through four 50 L Q5 HotStart reactions with index primers for 10 cycles. Final libraries (225 bp) were sequenced on Illumina platform with paired-end 50 bp reads targeting 50 million reads per library.

For barcode-variant mapping, the contiguous kinase-substrate-barcode segment (1.7 kb) was amplified using KAPA HiFi polymerase with 24 parallel reactions per kinase to preserve library complexity. Thermocycling parameters included 14 cycles with 10-second annealing at 71° C. and extension at 20 seconds per kilobase. Products were pooled, purified, and sequenced on the PacBio Revio platform, generating approximately 45 million HiFi reads across four SMRT cells.

### Next Generation Sequencing Analysis

Bioinformatics pipelines were developed to construct barcode-variant look-up tables from long-read PacBio sequencing and to measure barcode abundances in selection experiments from short-read Illumina sequencing.

Barcode-variant look up table construction. The goal of the analysis was to create a high-fidelity map between barcodes and their associated kinase variants from the long-read PacBio sequencing data. Due to sequencing errors, multiple reads from the same barcode may contain slight differences. We sought to determine the true underlying sequence for each barcode by using Bayesian statistics to integrate information across all reads with a common barcode. The first step in the analysis was to extract barcode and kinase variable region sequences from the PacBio reads. HiFi FASTQ reads from each kinase library were filtered for the exact vector context, the presence of both the DHFR-substrate cassette and a 3 overhang motif. Reads with either anchor missing were discarded. Reads that passed this QC step were parsed to extract the landing-pad barcode and the kinase ORF. We validated the kinase library identity via a two-base tag embedded in the barcode (AG, CT, and TA for Fyn, Lck, and Src, respectively); only tag-consistent reads were retained. ORFs were length-checked (765 nt), translated, and compared codon-wise to the kinase-specific wild type to perform nucleotide and amino-acid level variant calling. All of the reads were grouped by their barcode identifier, creating sets of reads that originated from the same original molecule. Each barcode group contains multiple, noisy observations of the same “true” underlying sequence.

The next step of the analysis was estimating the consensus variant sequence for each barcode with Bayesian statistics. For each position i in the variable region, the nucleotide bᵢ∈{A, T, C, G} that is most likely the true base is determined, given the observed data Dᵢ (e.g., all bases observed at position i in all reads with the same barcode). Bayes rule gives us

$$P ( {b}_{i} = b \vert {D}_{i} ) = \frac{P ( {D}_{i} \vert {b}_{i} = b ) P ( {b}_{i} = b )}{P ( {D}_{i} )} ,$$

where P(bᵢ=b|Dᵢ) is the posterior probability that the true base at position i is b given the observed data, P(Dᵢ|bᵢ=b) is the likelihood of observing the data if the true base were b, P(bᵢ=b) is the prior probability that position i contains base b (which we set to 0.25 for a uniform prior), and P(Dᵢ) is a normalization constant (equal to Σᵢ P(Dᵢ|bᵢ=b) P(bᵢ=b)) to ensure the probabilities sum to 1. To compute the position-wise consensus, at each position i the base with the highest posterior probability, e.g.,

$${\hat{b}}_{i} = argb ∈ {max}_{b ∈ \{ A , T , C , G \}} P ( {b}_{i} = b \vert {D}_{i} )$$

is selected. The maximum posterior probability at each position is used as a confidence score; an overall score for the complete consensus sequence was determined by computing the joint posterior probability assuming position-wise independence, P (consensus sequence correct)=ΠᵢP(bᵢ=\hat{b}ᵢ|Dᵢ). To ensure the accuracy of the lookup table, barcode-variant pairs with a joint posterior probability below 0.99 were filtered out and excluded from subsequent analyses.

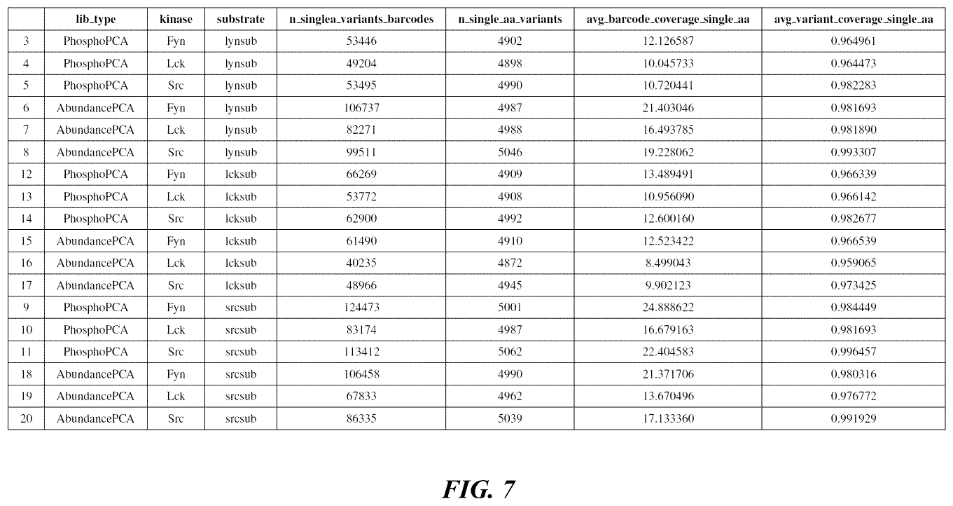

Bioinformatic analysis of deep mutational scanning data. Paired-end short-read sequencing data from selection experiments were processed using the MAGeCK (Model-based Analysis of Genome-wide CRISPR-Cas9 Knockout) pipeline to count barcodes. Paired-end FASTQ files were quality filtered and merged using fastp with a minimum overlap requirement of 6 base pairs. Barcode abundance was quantified using MAGeCK count, with 34 nucleotides trimmed from the 5′ end to remove constant upstream sequences. This analysis generated sample-labeled count matrices for both pre-selection and post-methotrexate selection replicates. For each screening condition, enrichment was assessed with MAGeCK ‘test’ by comparing pre- and post-selection replicates, using the -remove-zero flag. This yielded per-barcode log 2 fold changes and false discovery rate adjusted p-values at the single-barcode level. To link Illumina barcode counts with PacBio-derived barcode-variant assignments, MAGeCK outputs were processed through several quality control steps. True barcodes were extracted as sequences 5′ of the constant flanking anchor (GCCTAGGCAGCTATGACCATGATTACGCCAAGCG, SEQ ID NO: 1) and filtered by expected library tags (AG, CT, or TA for Fyn, Lck, and Src libraries, respectively). Only barcodes with minimum pre-selection coverage (>20 reads) were retained for analysis. Barcode sequences were matched to the PacBio lookup table using exact sequence identity. Analysis was restricted to barcodes corresponding to two or fewer amino acid changes. To remove outlier measurements, barcode-level log₂ fold changes were z-score normalized within each variant group, and barcodes with |z|>2 were excluded from further analysis. Final variant matrices were generated by collapsing barcode counts across wild-type, single-nucleotide synonymous, and single-amino-acid variants; these matrices were analyzed with Rosace to generate fitness scores for each variant. The resulting 18 screening libraries achieved a barcode-variant coverage of 10-25× and included between 95.9% and 99.3% of all possible single amino acid mutations within the 254-residue kinase domain (FIG. 7).

### Molecular Dynamics Simulations

Kinase-substrate complex structures were generated using Chai-1 in multimer mode with ligands and geometric constraints enabled, where each complex contained one kinase domain (Chain A), one substrate peptide (Chain B), two magnesium ions (Chains C and D), and ATP (Chain E), with MMSeqs2 and template matching settings applied. To ensure catalytically relevant conformations, we implemented specific geometric constraints including inter-chain contacts (4.0 Å distance) between A: R119 and B: Y8, and between A: D120 and B: Y8 to position the phosphorylatable tyrosine near the HRD motif, as well as pocket constraints (4.0 Å distance) between ATP (Chain E) and B: Y8, and between ATP and A: L127 to maintain proper positioning within the catalytic cleft. For molecular dynamics simulations, ATP and Mg²⁺ coordinates were refined by superimposing reference crystallographic structures onto the Chai-1 predicted models using PyMOL, with ATP positioning based on PDB structure 1QMZ and Mg²⁺ coordinates derived from PDB structure 1IR3. Molecular dynamics simulations were performed using GROMACS 2025.2 with the CHARMM36-jul2022 force field and TIP3P water model, where protein topologies and position restraints were generated using pdb2gmx, and simulation protocols included energy minimization, NVT equilibration, NPT equilibration, and 500 ns production runs under standard conditions. Trajectories were processed using trjconv to center molecules and maintain molecular integrity (-pbc mol-center-ur compact) and to remove periodic boundary condition artifacts (-pbc nojump), with additional PBC correction for individual protein chains performed using the MDAnalysis Python package when GROMACS processing was insufficient, and stable trajectory segments of 100 ns duration were identified based on root mean square fluctuation (RMSF) analysis and visually inspected using ChimeraX for selection as representative conformational states.

### Decoupling of Abundance- and Phospho-PCA Data Through Hierarchical Bayesian Modeling

Deep mutational scanning (DMS) experiments often confound several biological effects that-in an ideal world-should be carefully separated to extract meaningful insights. In the kinase studies presented herein, measured phosphorylation depends on protein folding stability, the probability the kinase is in an active state, and the specificity of the kinase-substrate interaction. Since protein misfolding reduces kinase abundance, which in turn decreases phosphorylation, folding stability acts as a confounder that must be separated from the other two effects. This is necessary to distinguish between mutations that genuinely alter substrate recognition or kinase activity versus those that simply impair overall protein stability.

The experimental design used herein employs a dual DMS screen to address this confounding issue, as abundance-PCA measures kinase folding and stability (e.g., the confounding factor) and phospho-PCA measures the kinase-substrate interaction (e.g., the target signal+confounder). The experimental data consists of abundance-PCA and phospho-PCA scores for each kinase identity (i), substrate identity (j), position along the kinase domain (k), and amino acid substitution (l). To decouple these effects, we developed a hierarchical Bayesian regression model that decomposes the observed phospho-PCA signal into three distinct components:

$$phospho - P C {A}_{i j k l} = {a}_{i j} \times {Abundance}_{i k l} + {b}_{i j} + {Activity}_{i k l} + Substrate {specificity}_{ijkl} ,$$

where each term captures distinct biological effects operating at different hierarchical levels of organization. This model architecture reflects the nested structure of biological variation through three different levels, each addressing different sources of experimental and biological variation. These levels are given below.
Level 1: Kinase-substrate experiment effects. At this level, we group mutations by each unique kinase-substrate pair (i and j) to capture overall biochemical context and experimental batch effects. This level also posits a linear relationship between abundance- and phospho-PCA scores. There are two key parameters at this level—aᵢⱼ, a slope parameter capturing how abundance affects phospho-PCA scores for each specific kinase-substrate pair, and bᵢⱼ, an intercept representing the baseline phoshporylation activity independent of abundance effects.
Level 2: Kinase activity effects. The second level addresses kinase activity effects by grouping mutations according to kinase identity, mutation position, and amino acid change (i, k, and l). This captures changes in kinase activity caused by mutations that are independent of substrate identity. The corresponding model term represents mutation-induced perturbations to kinase catalytic activity that affect all substrates equally, reflecting changes to the fundamental enzymatic machinery rather than substrate-specific recognition elements. For example, this term captures mutations that introduce a bias towards active or inactive conformations.
Level 3: Kinase-substrate specificity effects. At the finest resolution, we group by all four factors—kinase (i), substrate (j), position (k), and mutation (l) to capture effects where mutations differentially impact particular kinase-substrate pairs. This accounts for potential substrate-specific modulation of a mutations impact, and represents the true substrate recognition signal we aim to isolate from confounding abundance effects.

The model design used herein was guided by the intuition regarding which aspects of the experiment should affect which model terms. The abundance score quantifies how mutations alter the abundance of the kinase itself; given that phosphorylation activity is inherently influenced by kinase concentration, failing to account for these changes would confound measurement of kinase activity. The model used herein addresses this by incorporating abundance as a covariate with kinase-substrate specific slopes (aᵢⱼ), allowing each kinase-substrate pair to have its own abundance-phosphorylation relationship. This should (in principle) statistically separate true changes in enzymatic activity from abundance-driven changes, enabling more precise inference of the functional impact of mutations. The abundance score and activity score are conserved for a single kinase across multiple substrates, further regularizing the robustness of the inference.

To regularize parameter estimates while allowing the data to drive model conclusions, weakly informative priors were employed. Specifically, defined herein include:
Abundance effect slopes: aᵢⱼ~ HalfNormal(1), which constrains effects to be positive (which is expected biologically)
Baseline activity intercepts: bᵢⱼ~Normal(0, σ_{hyper}), which allows experiment specific baselines
Kinase activity effects: Activityᵢₖₗ~Normal(0, σ_{activity}), which is zero centered with a learned scale.
Substrate specificity effects: Substrate specificityᵢⱼₖₗ~Normal(0, σ_{specificity}), which is zero centered with a learned scale.

The hierarchical prior structure includes hyperpriors that regulate the baseline variability across different model components. The hyperparameter σ_{hyper} controls experiment-level variation, while σ_{activity} and σ_{specificity} control the magnitudes of biological effects at their respective levels. The σ_{hyper} and σ_{activity} scale parameters follow half-normal distributions with unit scale, while the σ_{specificity} scale parameter follows a half-normal distribution with ½ scale. This hierarchical prior structure allows partial pooling, encouraging shrinkage of weakly supported group effects while letting strongly supported signals emerge. Ultimately, this approach provides sufficient regularization while remaining sufficiently uninformative to prevent overfitting in sparse or unbalanced datasets. The model was implemented in PyMC and fit to the data using the No-U-Turn Sampler (NUTS). Convergence statistics (e.g., \hat{R} and effective sample size) were used to ensure sample quality.

### Example 2

Uncovering the Molecular Basis of Kinase Activity and Substrate Recognition with Phosphor-PCA

### Phospho-PCA Enables Deep Mutational Scanning of Kinase Domains

Phospho-PCA uses a growth-based biosensor to couple kinase-substrate interactions to the growth of budding yeast cells; a schematic is shown in FIG. 2. This biosensor consists of two parts—the first is a kinase domain tethered to its substrate and one-half of a split-dihydrofolate reductase (DHFR) enzyme. The second is a phospho-amino-acid binding domain (PAABD) tethered to the other half of the split-DHFR enzyme. If a kinase-substrate interaction occurs, the substrate is phosphorylated, leading to the binding of the PAABD to the substrate. This binding reconstitutes the DHFR enzyme, conferring resistance to methotrexate. It is noted that this construct design was inspired by fluorescent kinase biosensors, which use a similar mechanism to couple kinase substrate interactions to changes in fluorescence. By removing the PAABD, this construct also enables abundance-PCA measurements. To perform deep mutational scanning, a library of kinase variants (made with one-pot saturation mutagenesis) was first cloned into the kinase portion of the biosensor. Because kinase domains are too long (750-900 bp) for short-read sequencing, the library was stochastically linked to short-read nucleotide barcodes and long-read sequencing was performed to construct a lookup table. Short-read sequencing is used to measure the abundance of each variant after methotrexate selection.

To validate phospho-PCA, growth curves of budding yeast expressing the biosensor construct disclosed herein were measured for a collection of kinase-substrate pairs; the results are shown in FIG. 3. Significantly higher growth for interacting kinase-substrate pairs was observed than non-interacting pairs (FIG. 3, panel a). Both catalytically dead kinase domains and mismatched substrates were used to create non-interacting pairs. The experiments validated that the growth-based biosensors worked for the Fyn, Lck, and c-Src tyrosine kinases, a collection of serine-threonine kinases, and for a panel of phospho-amino acid binding domains (PAABDs) (FIG. 3, panels a, c). Importantly, by quantitatively extracting growth rates during the exponential phase of cell growth, Applicant observed that the biosensors accurately recapitulated the known preferences of Fyn, Lck, and c-Src for their respective substrates (FIG. 3, panel b). These results demonstrate that the growth-based biosensors can be utilized to quantify the strength of kinase-substrate interactions during cell growth.

With the growth-based biosensors validated, libraries were constructed to explore the molecular basis of substrate recognition for the SFKs Fyn, Lck, and c-Src. Each library paired all possible single-site variants of Fyn, Lck, and c-Src with one potential substrate (Fyn-sub, Lck-sub, and c-Src-sub). 18 biosensor libraries that covered phospho-PCA and abundance-PCA were constructed for all possible kinase-substrate pairs. These libraries were screened by measuring growth rates in the presence of methotrexate selection with short-read sequencing. In-depth descriptions of the library assembly method, analysis of each library's mutation composition and coverage, methotrexate selection, protocols, and bioinformatic analysis of the sequencing data are given in Example 1.

### Structural Context Determines the Impact of Mutations on SFK Stability

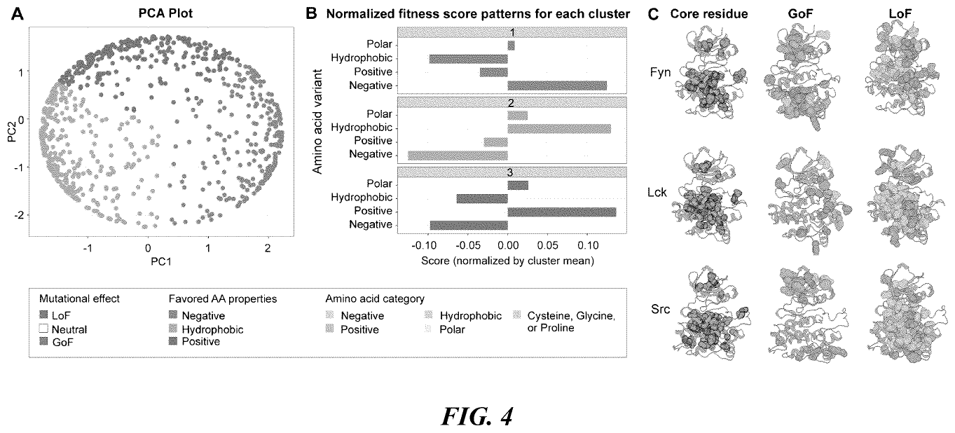

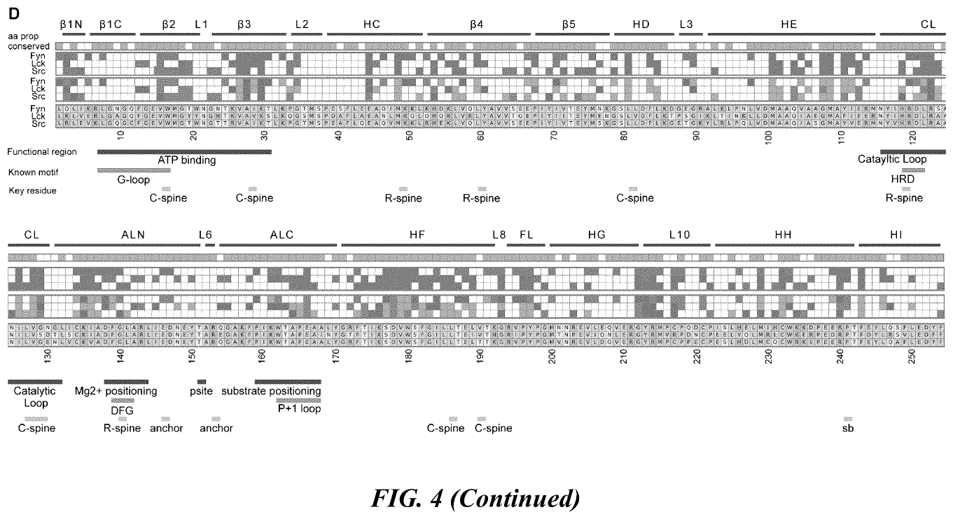

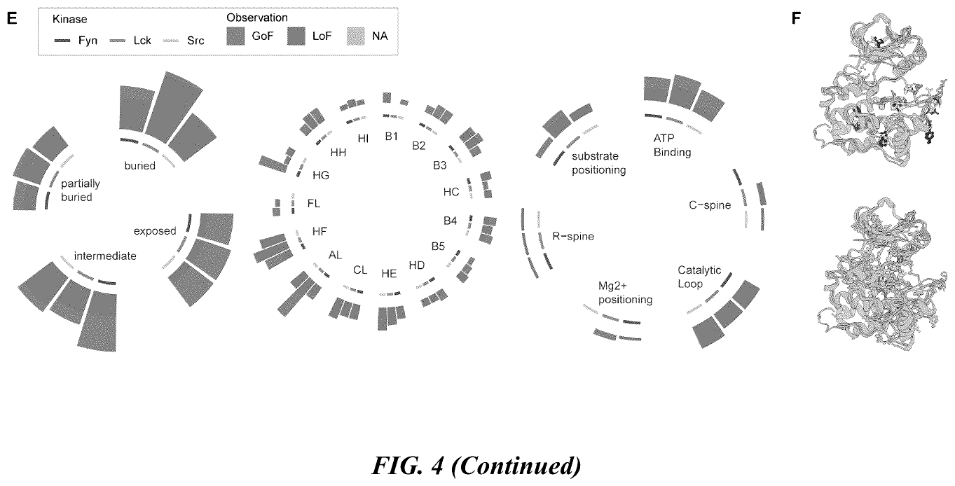

The analysis of the abundance-PCA experiments for Fyn, Lck, and c-Src kinases is shown in FIG. 4. To simplify the description of the results, the convention of Modi and Dunbrack was adopted to describe relevant sections of the kinase domain. This convention defines 17 key sections throughout the kinase domain-8 in the N-terminal domain (B1N, B1C, B2, B3, HC, B4, B5, HD) and 9 in the C-terminal domain (HE, CL-catalytic loop, ALN-activation loop N terminus, ALC-activation loop C terminus, HF, FL, HG, HH, HI). Each section is named for its dominant secondary structure or its functional role.

To investigate the impact of mutations on SFK folding stability from a biophysical perspective, a principal component analysis of the abundance-PCA data was performed. For this analysis, each amino acid (excluding proline, cysteine, and glycine) was first grouped into four biophysical categories: hydrophobic, polar uncharged, positively charged, and negatively charged. The biophysical category and abundance data were then compiled for all mutants across all positions and kinases, and principal component analysis was performed on the resulting matrix. The results are shown in FIG. 4, panels a and b. Using the second, third, and fourth principal components, three clusters were identified in which the presence of negatively charged (1), hydrophobic (2), or positively charged residues (3) leads to a gain-of-function effect in abundance-PCA, respectively, with alternative properties resulting in a loss-of-function effect. Next, this clustering was used in combination with physical and structural reasoning to explain the impact of convergent and divergent mutations (e.g., mutations with the same or different effects across all three SFKs, respectively). With this goal in mind, several observations were made. First, most of the conserved abundance loss of-function mutations occurred within the hydrophobic core, although a number of mutations also occurred in structurally distinct regions such as the HE and the catalytic loop. Regions with reduced solvent-exposed surface area (SASA, <10° A) were enriched for abundance loss-of-function mutations. In contrast, regions with more solvent exposure were enriched for gain-of-function mutations (FIG. 4, panel c). These observations were consistent with the clustering analysis; cluster 2 sites were located in the hydrophobic core, while cluster 1 and 3 sites were located at the surface (FIG. 4, panel c). The impact of mutations on abundance-PCA in functionally important, evolutionarily conserved regions (FIG. 4, panels d and e) were also analyzed. The results show that each functional region had a different mutational tolerance. The catalytic loop, which is important both functionally and structurally, and residues in the N-terminal portion of the activation loop responsible for magnesium positioning, were enriched with loss-of-function mutations. In contrast, the region responsible for ATP binding contained both gain-of-function (B2) and loss-of-function (B3) mutations.

Significant divergence was observed for abundance gain-of-function mutations across the three SFKs; these sites were enriched in HF-HG for Fyn, AL for Lck, and B2 for c-Src. The results also show that the sites for these divergent mutations overlapped with regions where the electrostatic charge distribution differed considerably between these three kinases (FIG. 4, panel d). A closer inspection of these locations revealed a consistent relationship between the physicochemical properties of amino acids and the phenotypic outcomes of mutations. Specifically, at several positions, the introduction of a charged residue that is compatible with the local electrostatic environment of one kinase led to a gain-of-function, while being neutral or detrimental to the other kinases that had a different charge distribution at that site. These sites include sites in cluster 1 (position 172 for Lck) and cluster 3 (positions 76, 88, 154, and 236 for Lck and Fyn). Similarly, divergent loss-of-function mutations also reveal location-specific preferences for electrostatic charge. These include mutations in cluster 1 (position 4 for Src) and cluster 3 (position 247 for Lck), suggesting that these kinases evolved to favor negative and positive charges at these positions, respectively.

Finally, the data show that networks of van der Waals interactions are a significant driver of divergence, as they impact the degree of solvent accessibility for specific residues and hence sensitivity to mutations. For example, at position 28, interactions present in Fyn and Lck bury a hydrophobic residue, leading to a loss of function when disrupted. In contrast, c-Src lacks these interactions, resulting in increased solvent exposure and increased tolerance to mutation. When examined together, these data present a consistent picture in which a residue's structural context determines the impact of mutation on SFK folding stability. Disruption of residues located within the hydrophobic core leads to reduced folding stability, while mutations that increase the opportunity for favorable solvent interactions improve stability. These effects can be brought about by directly changing the physicochemical properties of a residue or indirectly by disrupting van der Waals networks that control surface exposure. It is noted that these observations are consistent with prior work, which demonstrates that solvent interactions can enhance a protein's thermostability and solubility.

### A Hierarchical Bayesian Model Separates the Contribution of Kinase Stability, Activity, and Substrate Specificity to Phospho-PCA Scores

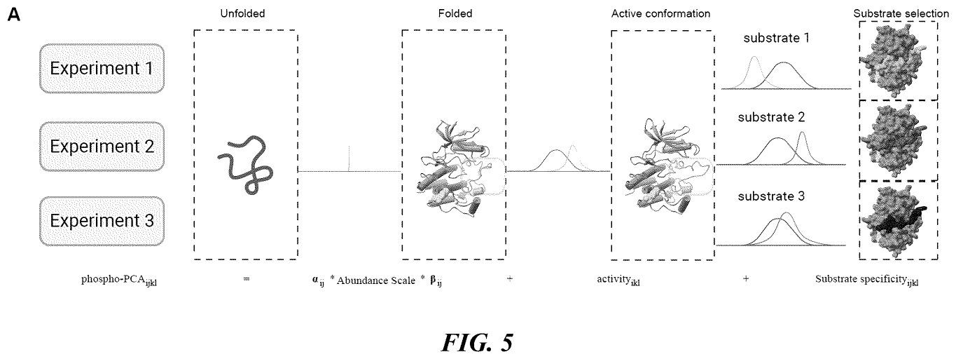

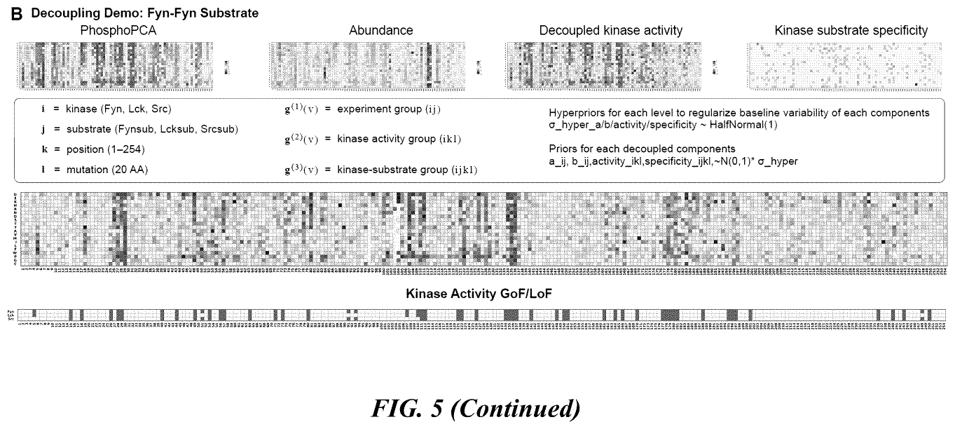

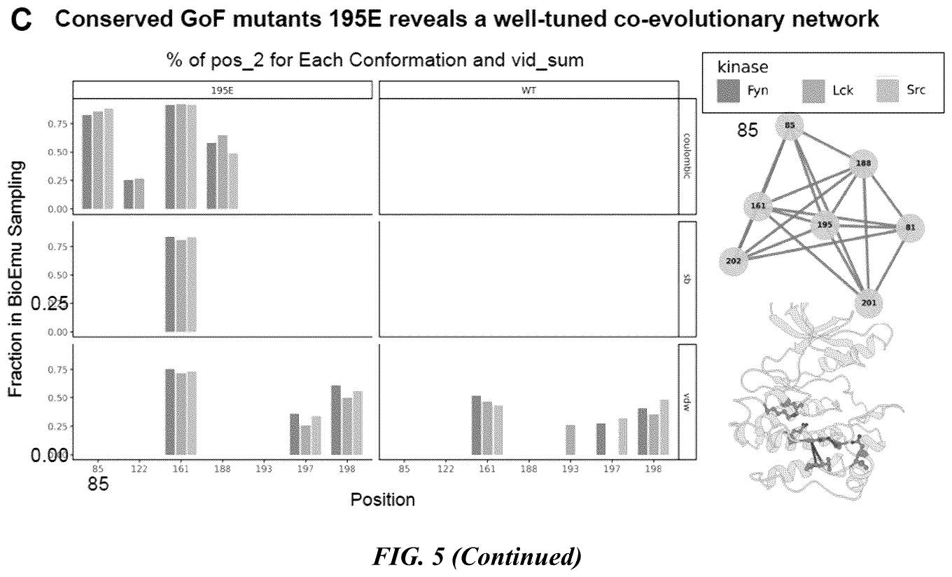

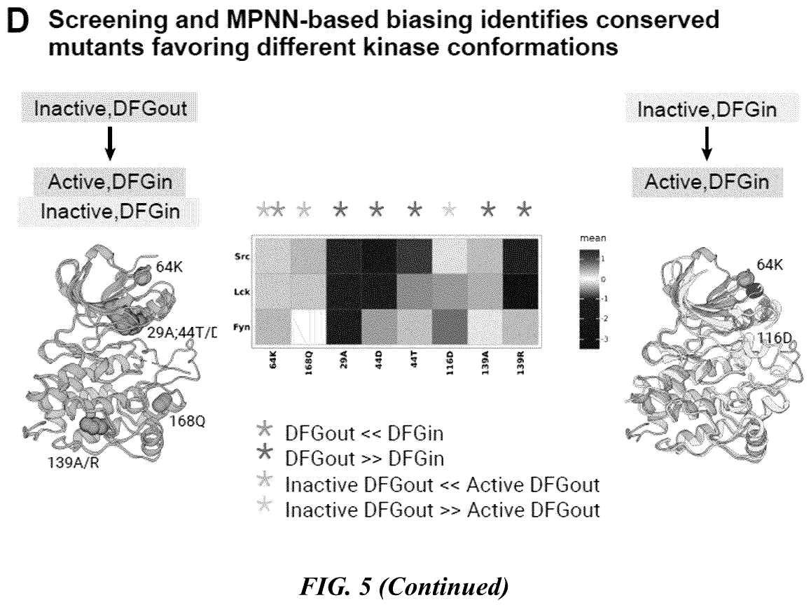

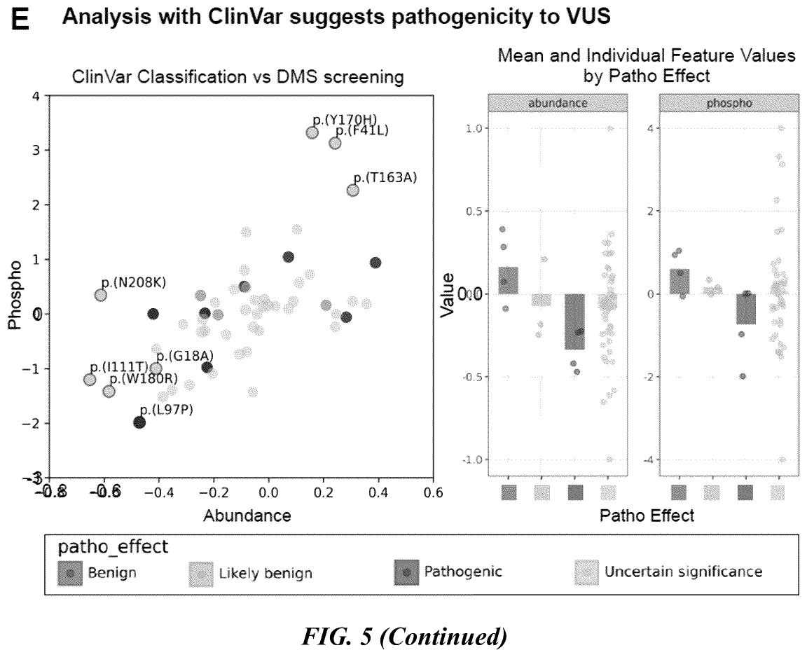

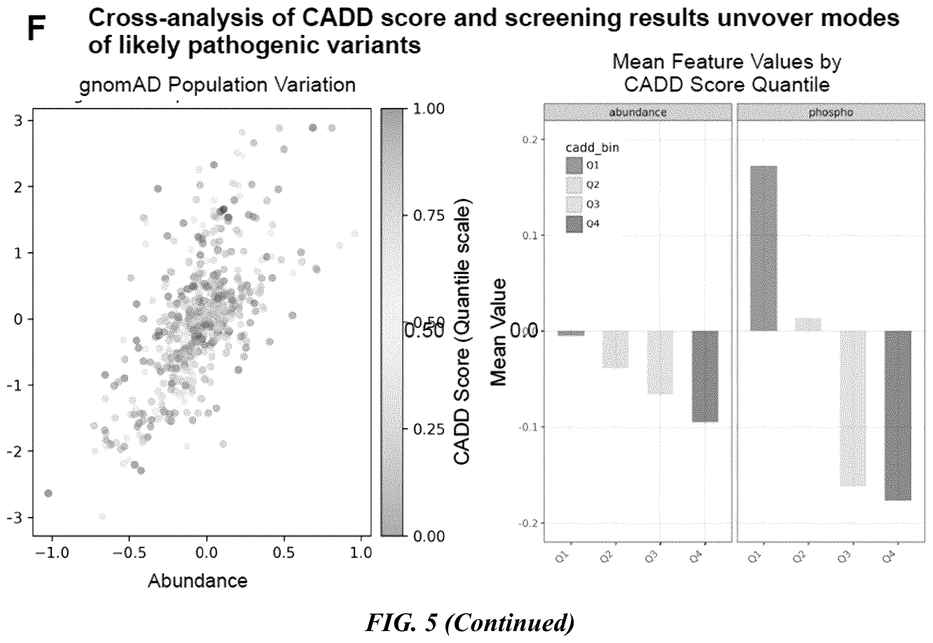

A significant challenge in understanding the impact of mutations on kinase function is the reality that each mutation has multiple pathways to perturb a protein's function. For kinases, mutations can impact the phospho-PCA score through three mechanistic pathways. First, a mutation could impact abundance, which would directly affect the phospho-PCA score (assuming rate-limited kinetics). Second, a mutation could promote a dynamic shift towards active or inactive conformations. Third, a mutation could change the preference of a kinase for a specific substrate. To understand the contributions of each of these possible mechanisms to the measured phospho-PCA scores, a hierarchical Bayesian model was developed. This model, displayed in FIG. 5, panel a and described in full detail in Example 1, posits that phospho-PCA scores can be decomposed into a linear sum of scores for a mutation's impact on abundance (derived from scaled abundance-PCA measurements), kinase activity, and substrate specificity. It is believed that some of these scores should be shared between conditions. For instance, scores for a mutation's impact on abundance and kinase activity should be shared across substrate identity. Scores for substrate specificity, however, are expected to vary among all four experimental variables (kinase, substrate, position, and mutation). This expectation can be viewed as a structural prior incorporated into the model for regularization. This model was fit to the data using Markov chain Monte Carlo; an example decomposition is shown in FIG. 5, panel b. One advantage of the Bayesian framework for modeling is its ability to quantify uncertainty, which enables the identification of key residues that influence kinase activity and substrate preferences. It is noted that this decoupling was made possible by the presence of the abundance-PCA data, which was a significant prior advance. Although deep learning-based methods for decoupling paired protein complementation assay data exist, it is found that the mutational signature of the libraries disclosed herein (e.g., heavily biased toward single-point mutations) and the need to separate the impact of three distinct mechanisms on the phospho-PCA scores required a bespoke modeling effort.

### Computational Decoupling Reveals Conserved Activity-Biasing Mutations

Heatmaps of decoupled scores for kinase stability, activity, and substrate specificity are shown in FIG. 5, panel b. Analysis of the decoupled phospho-PCA scores identified residues that primarily control SFK catalytic activity, distinct from those essential for abundance. Applicant reasoned that these mutations function either by directly impacting catalysis or by biasing the kinase toward specific conformations, and this hypothesis was used to structure the analysis.

Residues were categorized based on their conserved functional impact across Fyn, Lck, and c-Src. To do so, residues where a loss-of-function (LOF) phenotype was conserved in both abundance-PCA and the decoupled phospho-PCA scores were first examined. 15 of the 24 conserved LOF positions from the abundance screen also exhibited a conserved LOF impact on kinase activity, pointing to their dual roles in maintaining stability and catalytic function. Some of these map to well-known structural motifs, reinforcing existing mechanistic insights. For example, V25 and K28 (β3 strand) are critical for ATP binding, with K28 forming a key salt bridge with E44 (α helix). Other residues with known catalytic roles include G107 (HE helix) and E166 (ALC helix), which is involved in substrate positioning. The analysis also highlighted residues forming the hydrophobic catalytic spine (C-spine), including L80 (HD helix), L121, and I126 (Catalytic Loop), whose strong LOF phenotypes underscore the C-spine's architectural importance. Furthermore, D178, a component of the regulatory spine (R-spine), showed a strong LOF phenotype, affirming its critical role in stabilizing the active kinase conformation. Beyond these canonical motifs, the screen described herein identified previously uncharacterized but indispensable residues such as Y110 (HE helix), 1136 (ALN helix), a cluster on the HF helix (V179, W180), and F243 (HI helix), suggesting they have unappreciated roles in kinase structure and regulation.

To identify residues whose influence is confined to catalytic regulation, Applicant focused on positions exhibiting a conserved LOF phenotype exclusively in the phospho-PCAdata. These residues suggest a specialized role in modulating kinase activity or conformation rather than contributing to overall protein folding. Several of these residues are involved in catalysis, including G18 (82 strand) and 128 (83 strand) for ATP binding, A137 (xDFG motif) for Mg2+ coordination, and K161 and A164 (ALC helix) for substrate alignment in the P+1 loop. Notably, the HF helix, which the analysis identified as highly involved in conserved patterns, contains critical residues like E188. In PKA, the corresponding E230Q mutant crystallizes in an inactive state even with ATP present, highlighting its role in stabilizing ATP binding. Similarly, S181 on the αF helix helps position the catalytic loop for efficient phosphoryl transfer. The screen also revealed previously unannotated residues with activity-specific LOF effects. L56 and V57, located near the αC-β4 loop, likely stabilize the R-spine assembly, a key interface for maintaining an active conformation (36). Other newly implicated residues, including V71 (gatekeeper motif), E112, Y197, and L250, occupy helices in the C-lobe that shape the active-site cleft, suggesting they are important for the allosteric flexibility required for activation.

While LOF mutations were common, conserved gain-of-function (GOF) residues that offer insights into activity regulation were also identified. For example, at position 150 (Y419 in c-Src), mutations preventing phosphorylation can enhance catalytic activity in a single-domain context by avoiding competitive autophosphorylation (92). However, this site is indispensable for higher-order functions, such as dimerization and assembly of macromolecular complexes, which are critical for in vivo signaling. At position Y170, a known autophosphorylation site, the phospho-mimetic mutation Y170E was enriched with a positive phospho-only score. This suggests that introducing a negative charge may shift the kinase from a less catalytically favorable C-lobe binding mode to a more active conformation. Another novel GOF site, V195E, appears to stabilize the active state by forming a strong Coulombic interaction and a new hydrogen bond with K161 in the P+1 loop. This interaction likely neutralizes the positive charge of K161, a mechanism previously shown to increase Src activity. This interaction is further explored in the following section.

Finally, analysis of canonical catalytic motifs such as HRD, DFG, and APE revealed that they were enriched with mutations that had a mild effect on abundance but a drastic negative impact on the phospho-PCA score. This resulted in strongly negative phospho-only scores for mutations like p. (F139R), p. (E166N), and p. (D120P), confirming their critical and direct role in phosphorylation. In contrast, mutations in other motif residues, such as p. (P165H) and p. (H118Q), had strongly negative scores in both assays, indicating a mixed effect on catalysis and protein stability. This distinction highlights the method's ability to decouple mutations that purely disrupt catalysis from those that also compromise protein stability.

### AI-Enabled Structural Analysis Reveals Conformation-Biasing Mutations

To understand the structural basis of activity-biasing mutations, recently developed computational methods for conformation stability analysis were applied. These methods utilize the inverse folding model ProteinMPNN to predict a protein's preference for specific conformations. Here, ProteinMPNN was used to explore whether a given mutation would introduce a bias towards a specific kinase conformation. For this analysis, available crystal structures of c-Src and Lck kinases in active (Src: 1YI6, Lck: 3LCK), inactive DFG-in (Src: 2SRC, Lck: 3B2W), and inactive DFG-out conformations (Src: 20IQ, Lck: 6PDJ) were used. For each single amino acid mutation, the preference for inactive DFG-in versus inactive DFG-out conformations were computed, as well as active DFG in versus inactive DFG-in conformations. This analysis revealed a limited, but meaningful, set of mutations that consistently favor specific conformational states across both kinases. Mutations 29A, 44D/T, and 139A/R were predicted to favor the inactive DFG-out conformation, consistent with their negative decoupled phosphorylation activity scores in the experimental data. Conversely, mutation 116D was predicted to favor the DFG-in inactive state, also correlating with reduced activity scores. Notably, mutations 168Q and 64K were both predicted to strongly favor the DFG-in active conformation over the DFG-out inactive state. However, only 64K was additionally predicted to favor the active conformation over the DFG-in inactive state. This prediction aligns with the experimental observation described herein that 64K represents a novel gain-of-function mutation across all three SFKs, while 168Q shows mild loss-of-function effects.

The molecular basis of the V195E gain-of-function mutation was also investigated using evolutionary coupling analysis and equilibrium conformation sampling. Evolutionary coupling analysis applied to Fyn, Lck, and c-Src revealed a co-evolutionary network involving residues 81, 85, 161, 188, 195, 201, and 202. To understand how the V195E mutation affects this network, BioEmu (98), a recent method for accelerated generation of conformational ensembles for medium sized proteins, was used to sample 4,000 conformations for each wild-type kinase and their corresponding 195E mutants. Structural analysis of the generated ensembles revealed that the 195E mutation establishes new Coulombic interactions with residues 85, 122, 161, and 188. Most significantly, a salt bridge between residues 161 and 195 was prevalent only in mutant conformations, bringing these residues into closer proximity. To explore the impact on kinase activity, Kincore was used to classify conformations as active or inactive. When grouping these results by the presence or absence of this salt bridge, Applicant observed that conformations containing the 161-195 salt bridge showed increased fractions of active states: from 2.9% to 8.1% for Fyn, 0% to 2.7% for Lck, and 0% to 6.1% for c-Src. These results demonstrate that the V195E mutation enhances kinase activity by stabilizing active conformations through the formation of a critical salt bridge within a network of co-evolving residues.

### Abundance- and Phospho-PCA Scores Reveal the Impact of Clinical Variants

Analyzing the abundance- and phospho-PCA scores from the deep mutational scans together can provide insight into the variant-to-function relationship for SFKs observed in humans. To do so, Applicant first analyzed the behavior of known benign and pathogenic variants across the three SFKs from the ClinVar database within the experimental data (FIG. 5). Benign variants displayed near-zero to mildly positive abundance- and phospho-PCA scores, consistent with preserved protein function. In contrast, pathogenic variants frequently displayed significantly negative values in one or both metrics, highlighting the essential nature of SFKs to human biology. Several variants demonstrated predominantly negative abundance scores with minimal changes in the phospho-PCA score, indicating that reduced protein stability alone can drive pathogenicity. It is speculated that the non-catalytic function of SFKs may contribute to the mechanistic origin of this observation. Furthermore, several pathogenic variants exhibited significantly negative scores for both abundance- and phospho-PCA. Interestingly, these trends were used in combination with the experimental data to infer pathogenicity for variants of unknown function (VUS). For instance, the VUS variant N208K displays a pronounced reduction in abundance-PCA while the VUS variants I111T, W180R, and G18A display significant reductions in both abundance- and phospho-PCA.

To expand the analysis beyond Clinvar's SFK variant collection, population allele frequency data from the Genome Aggregation database (gnomAD) was collected and used to generate Combined Annotation Dependent Depletion (CADD) scores, a computational prediction of how harmful a genetic variant is likely to be. These CADD scores were then used to continue the analysis of the functional impact of SFK variants. When overlaid on the experimental data, where the abundance- and phospho-PCA scores were plotted for each variant, it was observed that variants with low CADD scores (presumably benign) clustered near the origin. However, high CADD variants were concentrated in the bottom-left quadrant. This was consistent with the analysis of the Clin Var database, which showed a link between variant pathogenicity and a negative effect on both scores. Interestingly, the top-left quadrant contained a smaller number of high-CADD variants with reduced abundance-PCA scores but high phospho-PCA scores. This suggests that these variants, which have a reduced frequency in the population, may be deleterious gain-of-function mutations.

### Computational Decoupling Reveals Substrate Specificity-Determining Variants

During the development of the hierarchical Bayesian model, it is observed that the specificity score was approximately an order of magnitude smaller than the other scores. This lower score reduced the model's ability to confidently distinguish true directional effects, resulting in increased uncertainty in the local false-sign rate. To address this limitation, the Bayesian model was supplemented with two additional approaches. First, a Kolomgorov-Smirnov test was used on the posterior samples for the specificity score to assess whether the posterior differed significantly from the prior distribution (e.g., the mutation has no impact on specificity). Second, stringent posterior probability thresholds at the 2% and 98% quantiles were applied. A mutation's specificity score was considered significant if it was deemed significant by both of these tests.

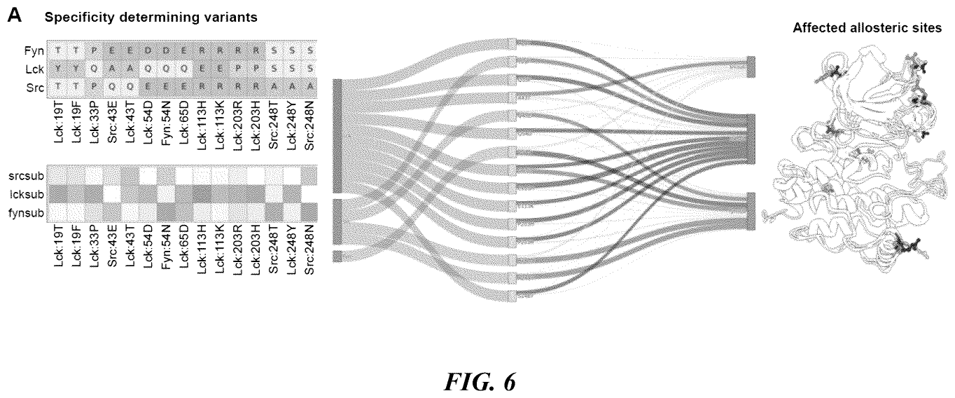

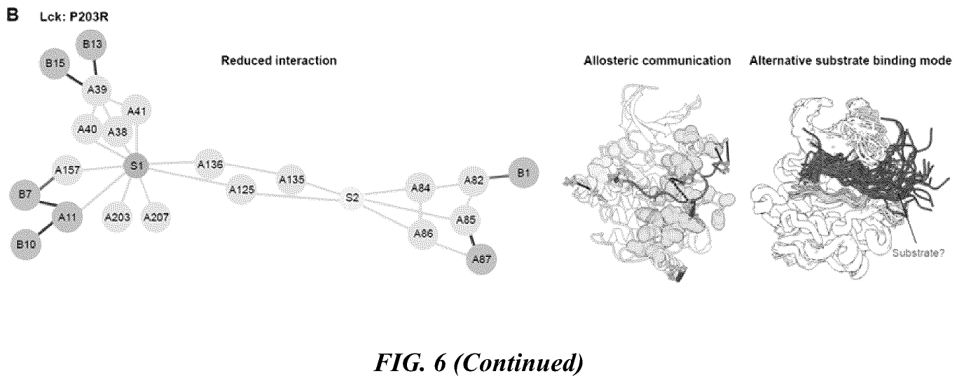

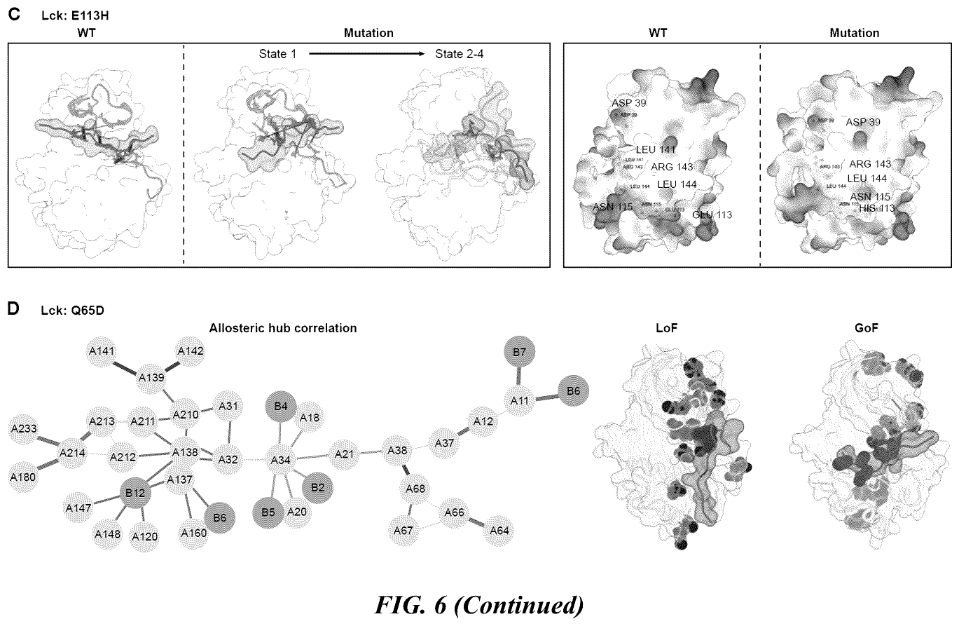

Next, Applicant sought to further identify mutations that induced a clear and directional preference change in the preferred substrate. To do so, amino acids were first categorized by their chemical property. Next, mutations where the mutant amino acid property matched the property of one kinase's wild-type property at that site, but differed for the others were identified. For these mutations, a significant increase in activity toward the matching kinase's substrate and/or a significant decrease in activity toward the wild-type substrate was observed. The results of the analysis are shown in FIG. 6; the identified mutations for each kinase were highlight below.

Fyn: The analysis revealed that the D54N mutation caused a loss-of-function towards the Fyn substrate by neutralizing the native negative charge at this position. This charge neutralization can disrupt electrostatic interactions that are optimized for Fyn-specific substrate recognition, which (if true) would demonstrate that precise charge distribution can significantly contribute to kinase-substrate pairing specificity.

Lck: Lck exhibited the largest collection of specificity-determining mutants, which may reflect its distinct evolutionary position among SFKs. The F19T mutation consistently decreased activity towards the Lck substrate, indicating that the native aromatic residue is essential for optimal substrate recognition. The Q33P mutation reduced the phosphorylation of the Lck substrate by replacing a flexible polar residue with proline, thus introducing structural constraints that likely impair the conformational flexibility required for substrate binding. The A43T mutation, which is adjacent to the critical E44 substrate-binding residue, introduces a polar hydroxyl group capable of hydrogen bonding and promoted activity toward the c-Src substrate. The Q54D mutation reduced activity towards the Lck substrate by replacing a neutral polar residue with a negatively charged one, which likely alters local electrostatics. The Q65D mutation has a similar impact on local electrostatics; in addition to reducing the activity toward the Lck substrate, it also increases the activity toward the Fyn substrate. The E113K and E113H variants, which exchange a negative charge for a positive charge, showed reduced activity toward the Lck substrate. The opposite charges present at this location between Lck and c-Src/Fyn, and the importance of this location for the preference of Lck substrate, suggest that this residue could be one of the important divergent residues separating the SrcA and SrcB subfamilies of SFKs. The results also show that the P203H and P203R mutations, which introduce a positive charge, reduced activity towards the Lck substrate. This observation was consistent with previous studies on SFKs; the impact of this location was further explored in the molecular dynamics study. Last, the S248Y mutation, which introduces a bulky aromatic tyrosine residue, reduced activity towards the Lck substrate.

c-Src: c-Src contained a collection of mutations that highlight the importance of surface charge and polarity. The mutation T19F drove preferential phosphorylation of the Lck substrate, complementing the Lck F19T findings and confirming that aromatic versus polar residues at this position provide a specificity switch between these kinases. At position 43, the Q43E mutation increased activity towards the Fyn substrate by introducing a negative charge that alters local electrostatics. Position 248 revealed a polarity-dependent specificity switch, as both A248T and A248N mutations increased activity towards the Fyn substrate by introducing a polar residue at a previously hydrophobic site.

Molecular Dynamics Reveals Allosteric Networks Governing Substrate Specificity that are Perturbed by Specificity-Determining Variants

The findings described above collectively demonstrate that substrate specificity arises from positioning residues with the appropriate physicochemical properties on the kinase surface. Because most of the residues were distal to the kinase-peptide binding interface (FIG. 6), it is believed that allostery was a key mechanism behind how these variants exert their effect on substrate specificity. To better understand allostery's role, a molecular dynamics study of several specificity-determining mutants was performed. To do so, a bound structure of the kinase and substrate with the Chai-1 protein folding model was first generated. To improve the accuracy of the bound model, additional distance constraints was included to ensure the substrate's tyrosine residue and ATP resided in the catalytic cleft; these are described in Example 1. Molecular dynamics was then performed with Gromacs of wild-type and mutant kinases in complex with their substrates for 500 ns.

For each simulation analyzed, the goal is to identify how the distal sites allosterically communicate with the kinase-substrate binding interface with a graph-theoretic approach. Briefly, a residue contact graph was first constructed from the data, where each residue is a node and nodes that form frequent contacts are connected with edges. These edges were further classified based on whether they were more (red) or less (blue) likely to be present in the simulation of a variant versus a wildtype kinase. Next step is to identify paths in this graph that link important residues. These residues include substrate residues with differing kinase contacts between the variant and wildtype, kinase residues that contact the substrate, and residues present in structural fragments with statistically significant differences in motion coupling between the variant and wildtype. An approximation of the Steiner tree problem was then solved to find a minimal subgraph of the residue contact graph that connects all important residues. A full description of the molecular dynamics analysis is given in Example 1. Strikingly, Applicant observed significant heterogeneity across the different variants with respect to how allostery mechanistically impacted the substrate preference. Some variants led to new binding modes, while others add new, inactive metastates to the kinase domain's conformational ensemble. The mechanism behind each specificity-determining variant analyzed herein is described below.

### Lck P203R Introduces a New, Inactive Binding Mode for the Lck Substrate

The simulation indicates that position 203 in Lck contains a critical allosteric site that controls substrate specificity through long-range communication networks within the kinase domain (FIG. 6, panel B). Previous work by Shah et al. identified the importance of this position in c-Src, where the corresponding residue (Arginine 472) assists in coordinating substrates that contain negative charges downstream of the tyrosine phosphorylation site. In their work, they noted that peptides that were selectively recognized by c-Src were enriched for sequences with positive charges downstream of the tyrosine. To begin the analysis of this site, it is first noted that this observation is consistent with the identification of Lck P203R as a specificity-determining variant described in the present disclosure. Lck P203R reduces Lck's affinity for its substrate while increasing activity towards Fyn/c-Src substrates. This effect correlates with the charge differences between the substrates; The Lck substrate contains significantly more positive charge and less negative charge than the c-Src substrate at positions +1, +7, +10, +13, and +15 relative to the phosphorylatable tyrosine. The introduction of a positive charge at position 203 likely introduces a preference for negative charges over positive charges at these positions, similar to c-Src.

To understand the mechanistic basis of this specificity switch, 500 ns molecular dynamics simulations were performed comparing wild-type Lck with the P203R variant. The simulations revealed dramatic changes in the substrate binding mode, with the mutant positioning the substrate closer to the C-lobe within the active site cleft and moving the phosphorylatable tyrosine further from ATP. This altered positioning was accompanied by specific changes in kinase-substrate interaction frequencies at the same peptide positions (+1, +7, +10, +13, and +15) that differ in charge distribution between Lck and c-Src substrates. Specifically, the P203R mutation caused both loss and gain of specific molecular interactions that explain the altered substrate positioning. Critical interactions were lost, including hydrogen bonding and salt bridge formation between kinase residue 39 and substrate position +15, as well as frequent contacts between kinase positions 11 and substrate positions +7 and +10, and between kinase position 39 and substrate position +13. Conversely, new interactions were established, including contacts between kinase residue 157 and substrate position +7, and significantly increased van derWaals and hydrogen bonding interactions between substrate position +1 and kinase residue 82.

The mutation at position 203 propagates its effects throughout the kinase domain through a sophisticated allosteric network. The arginine substitution introduces new intramolecular interactions, starting with sidechain hydrogen bonding between residues 203 and 207, and increased contacts between residues 205 and 197. These local changes significantly alter interaction frequencies along the 203-205-197-196-161-159 pathway, propagating the impact from the αG helix to the αF-αG loop—a flexible allosteric hub—and ultimately to the P+1 loop critical for substrate positioning. This allosteric communication extends further through regulatory spine interactions involving the catalytic loop and activation loop to reach the N-terminal lobe of the kinase domain.

### Lck E113H Creates New, Inactive Metastable States

Position 113 is a residue with divergence in its electrostatic properties between the three SFKs; Lck harbors a negative glutamic acid at this position, while Fyn and c-Src contain positive arginine residues. This position lies five residues upstream of the HRD motif within the catalytic loop, a location previously implicated in substrate specificity control. Shah et al. demonstrated that c-Src tolerates acidic residues near the phosphoacceptor more effectively than Lck, whereas Lck shows greater tolerance for positively charged contexts, indicating that charge features around the active site help determine which peptides are efficiently phosphorylated. Molecular dynamics simulations of the E113H mutation revealed that charge reversal at this position fundamentally alters Lck's tolerance for positively charged substrates by stabilizing non-productive binding conformations. Across two independent 500-nanosecond simulations, the E113H mutant consistently adopted four distinct metastable macrostates that positioned the Lck substrate in significantly different binding modes compared to wild-type Lck-substrate interactions. These alternative conformations represent phosphorylation-incompetent states that explain the reduced activity toward the native Lck substrate. Comparison of wild-type and mutant trajectories revealed specific molecular contacts that are disrupted by the E113H substitution. In wild-type Lck, frequent contacts between Nterminal activation loop residues, including Arg143-substrate position +12 and Leu141-substrate+11 (Proline), efficiently position the substrate within the catalytic cleft for optimal phosphoryl transfer. These stabilizing interactions were absent in the Lck E113H variant, resulting in altered substrate positioning that compromises catalytic efficiency.

Graph-theoretic analysis was used to understand how the perturbation at position 113 leads to the creation of these metastable macrostates with altered binding modes. In the analysis, the perturbation at position 113 propagates through a defined molecular pathway involving residues 113-115-144-143/141, ultimately affecting both the positioning of the activation loop and the organization of the αC helix. The αC helix, which is known from structural studies to organize the active site and participate in the conformational switch between the inactive and active states, showed altered interactions with the G-loop in the variant. Specifically, contacts between αC helix residues 40/41 and G-loop residue 12 were significantly reduced, as were interactions involving positive substrate residues at positions +5 and +10 with kinase residues 12, 10, and 40. These conformational changes resulted in the substrate adopting looser docking within the activation cleft, leading to the formation of alternative salt bridges that stabilize non-productive conformations. In metastates 2 and 4, new salt bridges formed between Arg143 and substrate Asp+9, while metastates 3 and 4 exhibited interactions between Asp120 and substrate Arg+5. It is speculated these alternative interaction patterns function as kinetic traps that sequester the substrate in catalytically incompetent conformations, explaining the reduced phosphorylation efficiency observed experimentally.

Lck Q65D Dynamically Sculpts the Substrate Binding Interface with Allostery

The Lck Q65D variant is a clear example of a specificity switch, as it causes a loss of preference for the Lck substrate while enhancing activity toward the Fyn substrate. The molecular dynamics study revealed that this variant's effect on substrate specificity arises from both altered surface electrostatics and communication through allosteric networks. The Q65D variant at the 84-65 turn introduces an aspartic acid residue at that position, which creates a new negative charge on the surface of the N-lobe near the substrate entrance that can electrostatically attract positively charged regions of substrate peptides. This effect explains, in part, the increased activity toward substrates like the Fyn substrate that multiple positive charges upstream of the phosphorylatable tyrosine, providing long-range cationic handles that enhance the encounter rates and binding affinity.

Additionally, the Q65D mutation affects substrate selectivity by modulating the kinase's preference for hydrophobic residues at the-1 position relative to the phosphorylatable tyrosine. Both Lck and c-Src favor bulky hydrophobic residues (Ile/Leu/Val) at this position, with the Lck substrate containing leucine and the Fyn substrate containing threonine. The mutation in the β4-β5 loop alters the allosteric networks that coordinate αC-helix/β-sheet coupling and substrate-nucleotide cooperativity, leading to new kinase-substrate interactions that are favorable to polar residues at the −1 position. Specifically, the Q65D mutation increased contacts between residues 64 and 66 while weakening interactions between residues 68 and 38. This local perturbation propagated to enhance contacts between residues 37 and 12, effectively pushing the αC-helix towards the G-loop and promoting more frequent coordination between residue 11 and substrate positions −1/0.

### Discussion

This example presents phospho-PCA, a new method that couples kinase-substrate interactions to the growth of budding yeast, which enables quantitative measurements of kinase-substrate interaction strengths. The example demonstrated that this method works for both tyrosine and serine-threonine kinases, demonstrating its applicability to this entire enzyme category. Phospho-PCA was used to perform deep mutational scanning of three kinase domains from the Src family, systematically evaluating the impact of thousands of mutations on specific kinase-substrate interactions. Pairing these data with deep mutational scans that measure the impact of mutations on kinase abundance allowed us to generate comprehensive maps of how sequence variation impacts kinase function with a resolution that would be difficult to achieve with conventional biochemical approaches. The example demonstrated that scores from the abundance- and phospho-PCA measurements can explain the functional impact of clinically significant variants, providing a framework for interpreting variants of unknown significance in clinical settings for this enzyme category. To better understand the contributions of each mutation to kinase abundance, activity, and substrate specificity, a hierarchical Bayesian model was developed to decouple the abundance- and phospho-PCA measurements. The decoupled scores allowed us to identify kinase variants with significantly altered activity and substrate specificity. Combining simulations of conformational ensembles with inverse folding models provided a mechanistic explanation for conserved variants that significantly altered kinase activity. A common theme among variants that impact substrate specificity was allostery, as mutations with a strong impact on kinase function frequently occurred away from the active site. Through molecular dynamics, it is possible to map the long-range allosteric networks that are responsible for these variants' impact on substrate choice.

This work has important implications for kinase biology, variant-to-function studies, and the AI/life science intersection. First, the study directly addresses how kinase sequence and structure influence both kinase activity and substrate preferences. By exploring this sequence-function relationship for three SFKs and their variants, one can gain a mechanistic understanding of how variants can shift the kinase's conformational ensemble to change their activity and how allostery can shape the kinase-substrate interface. In particular, it is noted that exploration of protein dynamics was essential to rationalize the observations. The insights gained from this study are likely important for the broader enzyme category. Second, understanding the variant-to-function relationship is essential for interpreting genetic variation in human populations. Due to errors in DNA replication, proteins in the human population exist as a cloud in sequence space rather than a single point. Characterizing the functional consequence of this variation is necessary to understand the genetic basis of human disease. The work described here provides a scalable framework for mapping these functional landscapes for protein kinases. Third, the work highlights the limitations of protein language models' ability to understand protein function. Although self-supervised learning approaches have shown remarkable progress in predicting protein properties, memorization and interpolation are their primary mode of reasoning. As a result, their performance is limited for proteins with limited data (e.g., variants) and complex phenomena such as allostery. Comparison of existing AI methods for identifying allosteric sites with protein language models with the data described herein showed a substantial lack of predictive power. This is expected, as there is a significant difference between the self-supervised learning signal used for model training and the functional properties of proteins that are of interest. The present work addresses this need by demonstrating a scalable approach to generating data on protein function. Combining such functional and biophysical information with protein language models may provide a promising path forward for this space.

It is worth noting that yeast is used as the model organism for phospho-PCA. The cellular environment (e.g., pH, ionic strength, and co-factor concentrations) in yeast differs from that in mammalian cells, which could impact the translation of the results. Further, yeast express a number of endogenous serine-threonine kinases; this background signal may pose a challenge for future phospho-PCA measurements. Bayesian approach is used to decouple abundance- and phospho-PCA scores. This model assumes that phospho-PCA scores can be decomposed linearly into abundance, activity, and specificity components, which may oversimplify the relationships between these factors. The analysis of clinical variants relies on a connection between the functional scores measured in yeast and human disease phenotypes. Future work should seek to validate this connection further, as this link likely depends on cellular and tissue contexts. The present work is focused on the kinase domain itself, but can be extended to perform deep mutational scans on the full-length kinase.

The growth-based biosensor described herein works for major kinase families, and the method requires only knowledge of kinase-substrate interactions to perform deep mutational scanning. The dataset provided using the methods described herein would provide insights into information transmission in signaling pathways, link variants of unknown function to human disease, provide broader insights into allosteric network organization, and facilitate the development of novel therapies targeting allostery. Such a comprehensive functional map would represent a significant advance in the understanding of how sequence variation shapes cellular signaling networks.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A phosphorylation-protein complementation reporter system, comprising:
a first fusion protein comprising a kinase substrate linked to (i) a kinase domain and (ii) a first segment of a reporter molecule; and
a second fusion protein comprising a phosphor-amino-acid binding domain (PAABD) linked to a second segment of the reporter molecule,
wherein the first segment and the second segment of the reporter molecule are capable of complementing one another to form an active reporter molecule via the interaction between the PAABD and the kinase substrate upon phosphorylation by the kinase domain.

2. The phosphorylation-protein complementation reporter system of claim 1, wherein the kinase domain is a tyrosine kinase or a portion thereof or a serine-threonine kinase or a portion thereof, optionally the kinase domain is a Src family kinase (SFK) or a portion thereof.

3. The phosphorylation-protein complementation reporter system of claim 1, wherein the kinase domain is Fyn, Lck, or c-Src.

4. The phosphorylation-protein complementation reporter system of claim 1, wherein the PAABD is selected from the group consisting of: a Src homology 2 domain, a PTB domain, a 14-3-3b protein domain, a WD40 domain, a WW domain, a forkhead-associated FHA domain, or a combination thereof.

5. The phosphorylation-protein complementation reporter system of claim 1, wherein the substrate comprises tyrosine, serine, threonine, or a combination thereof.

6. The phosphorylation-protein complementation reporter system of claim 1, wherein the reporter molecule is an inhibitor-resistance conferring protein.

7. The phosphorylation-protein complementation reporter system of claim 1, wherein the kinase substrate is linked to i) the kinase domain and (ii) the first segment of the reporter molecule via a linker.

8. The phosphorylation-protein complementation reporter system of claim 1, wherein the phosphor-amino-acid binding domain (PAABD) is linked to the second segment of the reporter molecule via a linker.

9. The phosphorylation-protein complementation reporter system of claim 1, wherein the reporter molecule is dihydrofolate reductase (DHFR).

10. The phosphorylation-protein complementation reporter system of claim 9, wherein the DHFR is a methotrexate-resistant DHFR.

11. An expression vector comprising a polynucleotide encoding the phosphorylation-protein complementation reporter system of claim 1.

12. The expression vector of claim 11, wherein the polynucleotides encoding the first fusion protein and the second fusion protein are on a single plasmid.

13. The expression vector of claim 11, wherein the kinase domain comprises a variation, optionally a substitution, a deletion, and/or an insertion, relative to its wild type sequence, and the polynucleotide further comprises a variant barcode associated with the variation.

14. The expression vector of claim 13, wherein the variant barcode is about 4-20 bp in length.

15. An abundance protein complementation reporter system, comprising:
a fusion protein comprising a kinase substrate linked to (i) a kinase domain and (ii) a first segment of a reporter molecule; and
a second segment of the reporter molecule,
wherein the first segment and the second segment of the reporter molecule are capable of complementing one another to form an active reporter molecule.

16. The abundance protein complementation reporter system of claim 15, wherein the second segment of the reporter molecule is expressed at a higher level than the fusion protein.

17.-20. (canceled)

21. An expression vector comprising a polynucleotide encoding the abundance protein complementation reporter system of claim 15.

22.-24. (canceled)

25. A method of detecting phosphorylation, comprising:
introducing into a cell an expression vector comprising a polynucleotide encoding a phosphorylation-protein complementation reporter system of claim 11;
culturing the cell in a selective culture medium under a condition for a time period, wherein the selective culture medium contains an inhibitor that inhibits cell proliferation in the absence of the active reporter molecule; and
detecting a cell proliferation signal, wherein the cell proliferation signal indicates phosphorylation of the kinase substrate by the kinase domain.

26.-29. (canceled)

30. The method of claim 25, further comprising:
introducing into a cell an expression vector encoding an abundance protein complementation reporter system of claim 21,
culturing the cell in a selective culture medium under a condition for a time period, wherein the selective culture medium contains an inhibitor that inhibits cell proliferation in the absence of the active reporter molecule; and
detecting an abundance cell signal.

31.-34. (canceled)

35. A method of screening kinase variants, comprising:
generating a kinase variant library comprising a plurality of expression vectors each comprising a polynucleotide encoding a phosphorylation protein complementation reporter system of claim 1, wherein the kinase domain comprises a variation and the polynucleotide comprises a variant barcode associated with the variation;
introducing into a cell an expression vector of the plurality of expression vectors;
culturing the cell in a selective culture medium under a condition for a time period, wherein the selective culture medium contains an inhibitor that inhibits cell proliferation in the absence of the active reporter molecule;
extracting expression vectors from surviving cells; and
determining the variation in the kinase domain by sequencing the extracted expression vectors.

36.-47. (canceled)
